(19) United States
(12) Patent Application Publication
(10) Pub. No.: US 2011/0008317 A1
(43) Pub. Date: Jan. 13, 2011

(54) CHARCOALS

(75) Inventors: Franciscus Antonius Anna Maria De Leij, West Sussex (GB); Tony Richard Hutchings, Hampshire (GB); Jeremy Robert Wingate, Essex (GB)

Correspondence Address: FOLEY & LARDNER LLP, P.O. BOX 80278, SAN DIEGO, CA, 92138-0278, US

(73) Assignees: The Forestry Commission; The University of Surrey

(21) Appl. No.: 12/671,686

(22) PCT Filed: Jul. 31, 2008
(86) PCT No.: PCT/GB2008/002612

§ 371 Date: Sep. 23, 2010

(30) Foreign Application Priority Data
Aug. 2, 2007 (GB) 0715050.1

(51) Int. Cl. A61K 8/19; C01B 31/08; A01K 29/00; C02F 1/42; B09C 1/08

(52) U.S. Cl. 424/125; 502/416; 119/28.5; 210/681; 210/502.1; 405/128.7

(57) ABSTRACT

Non-activated charcoals from living plant materials are useful as ion exchange agents for adsorbing cations from an environment, especially metal ions.

The present invention relates to charred organic materials useful in remediation of substances and conditions having metal contamination.

Adsorption of metals onto, adsorbents is known, and products on the market that are effective at removing metals from solutions include zeolites, red clays, ion exchange resins, bone charcoal and fungal biomass.

Zeolites are probably the most widely used product for metal removal from waste water. Zeolites can be natural or synthetic, the latter being able to adsorb around 10× more metal ions than natural zeolites. Metal adsorption capacities onto synthetic zeolites are as follows: (Cr)=0.838 mmol/g, (Ni)=0.342 mmol/g, (Zn)=0.499 mmol/g, (Cu)=0.795 mmol/g, (Cd)=0.452 mmol/g while natural zeolites adsorb: (Cr)=0.079 mmol/g, (Ni)=0.034 mmol/g, (Zn)=0.053 mmol/g, (Cu)=0.093 mmol/g, (Cd)=0.041 mmol/g.

Charcoals made from bone are well known for their ability to adsorb heavy metals and are widely used by industry to remove metals from solutions. Their potential to adsorb metals is similar to that of synthetic zeolites. The mechanism by which bone charcoal adsorbs metals is thought to occur via the formation of metal-phosphates. Bone consists mainly of apatite [Ca₁₀(PO₄)₆(OH)₂]. After charring, the phosphate groups that are present on the charcoal surface when coming into contact with metal ions are thought to form metal phosphates that are very stable, even at low pH. Materials high in phosphate are often used to immobilise heavy metals. Phosphate sources that have been investigated to immobilise heavy metal ions include: soluble phosphate salts, rock phosphate, synthetic hydroxyapatite, bone meal and phosphatic clay (Knox et al., 2006). Charcoal produced from chicken litter can also adsorb heavy metals via the formation of metal phosphates (Lima and Marchall, 2005).

Charcoal is formed from the partial pyrolysis of carbon-rich organic materials under non-oxidising conditions (Paris et al., 2005). In particular, charcoal is usually made from the xylem, especially the secondary xylem, of woody plants, being the “dead” portion that is processed into timber for instance.

In general charcoals are porous and their adsorbing properties are often related to the large specific surface area within the charcoal. During the charring process, most of the chemical bonds in the starting material are fractured and rearranged, leaving a surface that contains many functional groups such as hydroxyl, carboxyl and carbonyl groups (Antal and Gronli, 2003). The adsorbing properties of charcoal can be further improved by a process of activation, involving partial oxidation of charcoal with carbon dioxide, steam, or acid at high temperature, to give a greater surface area per gram charcoal that consists largely of graphene layers (Baird and Cann, 2005; Machida et al., 2005). Metal cations will adsorb at specific surface sites that have acidic carboxyl groups (Iyobe et al., 2004; Machida et al., 2005). These surface functional groups enable the binding of cations, including heavy metal ions. However, commercially available activated charcoals made from wood are in general not particularly good at binding metals. We found adsorption of copper onto activated charcoal never to be higher than 5000 mg/kg.

Fungal biomass has been used to immobilise metals, with maximum metal absorbance of 43,000 mg/kg biomass being reported by Niyogi et al. (1998) for Rhizopus arrhizus. Fungal biomass is liable to degradation, resulting in the subsequent release of any bound metals. The stability of the binding will depend on the functional groups that are present on the biomass and include chitin, amino, carboxyl, phosphate and sulphydryl groups (Norris and Kelly, 1977; Tobin et al., 1990).

There is a need to provide materials capable of adsorbing metals that overcome one or more of the above disadvantages. In particular, there is a need to provide materials that are relatively easy and/or cheap to produce. It is a further object to use renewable resources. It is also an object for the materials to be non-degradable. We have surprisingly found that charcoals produced from the shoots and leaves of fast growing plants as well as algae are capable of adsorbing large amounts of heavy metal ions from solutions and are capable of meeting one, some, or all of the above identified objects. The algae may be micro algae, but macro-algae are particularly preferred.

Mechanisms to improve adsorption of metal ions by known, woody charcoals have been proposed, such as oxidation of the “aromatic carbon backbone of the charcoal,” while creation of a larger surface area could further enhance the exposure of negatively charged carboxyl groups. In contrast, we have surprisingly discovered that charcoals derived from living plant material, such as young bark or foliage, as distinct from the xylem of woody plants, and dead bark, can, in fact, adsorb a large amount of metal ions, from a selected environment, such as a brown field site or polluted soil, slurry or solution, for instance, via ion exchange mechanisms. What is particularly surprising is that the mechanism for this has been shown to be completely different from that proposed previously. The present inventors have discovered that metal adsorption by charcoal produced from plants of all kinds is actually via uptake of the pollutant metal ions and exchange of said pollutant ions with pre-existing ions contained in the charcoal. In particular, potassium, calcium and/or magnesium ions that are present in the charcoal are exchanged for the pollutant metal ions, such as copper, thus completely removing the pollutant metal ions from the selected environment.

Activation of charcoal to produce activated charcoal is known in the art, achieved for instance by application of steam, carbon dioxide or acid, at high temperatures. This is a costly process requiring further steps and substrates as well as lots of energy. Surprisingly, However, we have shown that activation is not necessary in order to provide adsorbent charcoal having, the ability to adsorb cations and in particular, heavy metal cations.

Thus, in a first aspect, the present invention provides an ion exchange agent for adsorbing cations, the agent comprising charred material, wherein the charred material is not activated and is produced from living plant material.

The charred material adsorbs cations, most preferably heavy metal ions. Preferably, the living plant material is foliage. The living plant material may be referred to as non-woody living plant material, which excludes charcoal produced from woody xylem or charcoal comprising pyrolysed woody xylem. In other words, the charred material is not made from ‘wood.’ Wood is hard, fibrous, lignified structural tissue produced as secondary xylem in the stems of woody plants. Wood is dead plant material. The plant material can be referred to as ‘bio-char’ or ‘agri-char,’ which are distinct from charcoal that is produced from ‘wood.’

Generally, it is preferred that the material may be parts of plants, rather than the whole plant. Preferred parts are bark, stems, shoots and foliage. Roots are not preferred. Preferably, the charred material is produced from living plant tissues that are less than three years old, more preferably less than 2 years old, more preferably less than one year old and even more preferably less than 6 months old at the time of harvest or collection.

The living plant material is preferably not dead material at the time of harvest or collection, such dead material preferably including wood or the dead portions thereof. Instead, it will be understood that the agent can, in some embodiments, include material other than living plant material. In other words, the agent can also include non-living or “dead” plant material, such as material that is metabolically inactive at the time of harvesting. Straw and dead stems of non-woody plants are also preferred. In certain embodiments, it may be useful to include charcoal produced from dead plant material, such as wood, in addition to the charcoal from living plant material.

It will be appreciated that the living plant material refers to tissues such as young metabolically active bark in woody plants and foliage in woody and non-woody plants, in particular. However, it will also be understood that this term includes all growing parts of the plant, for instance those that were “active” or alive at the time or shortly before the plant was processed, dried, cut down, harvested or charred. It is particularly preferred that the material is metabolically active at the time of harvesting. Preferably, the material is non-xylem material, preferably not secondary xylem material.

In other words, it is preferred that the living tissue can be considered to be metabolically active (alive) at the time of harvesting, before drying and/or processing to charcoal. It will be appreciated that living plant material also preferably excludes core wood and old bark, despite the fact that these tissues originally consisted of cells that were once alive, in the sense of being metabolically active. These cells have, at the time of harvesting the plant material, died or substantially ceased metabolic activity.

It will be appreciated that bark is formed according to similar principles as wood, with new layers being added each year, in much the same way as the “year rings” in wood. The younger bark is found towards the radial centre of the plant, with older bark forming the outer surface. Preferably, the living plant material is living bark.

Preferably, this is around 1 year or less old, although it will be appreciated that the transition from living to dead is a gradual process.

Therefore, it is preferred that the living material is parts of the plant that had a recent active metabolism at harvesting. It will be readily apparent to the skilled person which tissues are alive and which tissues are dead.

The xylem, particularly the secondary xylem, of woody plants is preferably excluded from the living plant material. Such tissue is often simply called “wood” and can be considered to be the portion of a woody plant that is processed into timber, for instance.

Furthermore, it will be understood that the living plant material can be “killed”, in the sense that it ceases metabolic activity, once harvested. In particular, it is envisaged that the living plant material can be harvested and dried and then turned into charcoal. Accordingly, straw and dried plant materials are preferred embodiments of the present invention. In the case of non-woody plants, the whole of the plant can be considered as comprising growing material. Therefore, in particularly preferred embodiments, the source material is nettle, beet, oilseed rape or seaweed and, therefore, the whole of the plant, except roots, can be used to provide the charcoal according to the present invention.

In woody plants in particular, it will be appreciated that the living plant material excludes the highly lignified tissues, such as the xylem mentioned above. Therefore, it is preferred that the living plant material excludes so-called “structural” material, which provides the woody plant with the majority of its structural framework for supporting itself.

The living plant material preferably excludes metabolically inactive wood taken from the core of the trunk or branches of a woody plant, although the present ion exchange agent may comprise some charcoal from such dead sources. Therefore, in some embodiments, it is preferable to remove dead plant material prior to harvesting, whilst in other embodiments this may not be necessary.

As used herein, the term ‘living plant material’ relates to those portions of a plant which, in vivo, have, or would be expected to have, an active metabolism, such as leaves, bark and stems. Preferred living plant material is selected from those portions of the plant occurring above ground.

In its most common meaning, “wood” is the secondary xylem of a woody plant, which is a heterogeneous, hygroscopic, cellular and anisotropic material. Wood is gereally composed of fibers of cellulose (40%-50%) and hemicellulose (15%-25%) held together by lignin (15%-30%). Preferred examples of woody plants are trees and shrubs. The portion of the plant above normal ground level when the paint is growing in its natural environment, i.e. foliage comprising the stem, branches, leaves and so forth, but not the roots (being below normal ground level) is preferred.

In an alternative aspect, the present invention provides an ion exchange agent comprising charred, non-lignified, plant material

As far as woody plants are concerned, particularly preferred plant materials or parts are young bark and foliage.

For woody and non-woody (herbaceous) plants, foliage primarily consists of the leaves of the plant, but may also include the stems and leaf stems.

Non-woody plants are often called herbaceous plants and have leaves and stems that die at the end of the growing season to the soil level. A herbaceous plant may be annual, bi-annual or perennial. Herbaceous perennial plants have stems that die at the end of the growing season. New growth forms from the roots or from underground stems or from crown tissue at the surface of the ground. Examples include nettles, bulbs, Peonies, Hosta and grasses. By contrast, non-herbaceous perennial plants are woody plants which have stems above ground that remain alive during winter and grow shoots the next year from the above ground parts, including trees, shrubs and vines.

Thus, in one embodiment, the plant is preferably a woody plant, for instance a non-herbaceous perennial. In this instance, the material is not wood and is most preferably bark or foliage.

In an alternative embodiment, the plant is preferably a non-woody plant, i.e. a herbaceous plant. In this instance, the material is most preferably foliage or stems.

It is also preferred that the plant material is from a herbaceous plant or a crop, such as rape and most preferably a Chenopodiaceae, such as a beet, particularly sugar beet, Beta vulgaris subsp. maritima (Sea Beet), Beta vulgaris subsp. vulgaris or Beta vulgaris subsp. cicla (Swiss Chard, Silverbeet, Perpetual Spinach or Marigold), spinach, beetroot or garden beet. Other beets, are also preferred, of course.

Also preferred are nettles, cabbage, garlic, bracken (especially the leaves), horsetail and crops such as cereals, rye grass and oil seed rape. Preferably, The plant may be a dicotyledon, although this is generally not preferred.

In other embodiments, the living plant material may be referred to as “young growth”. In relation to woody plants, in particular, such growth can be considered to be less than one year old.

As referred to above, particularly preferred examples of non-woody plants are the foliage and stems. Particularly preferred examples for woody plants are bark and foliage. In both cases, the foliage is particularly preferred. An advantage of the present invention is that such foliage is often discarded during more industrial processes such as preparation of timber or farming of crops such as sugar beets, for instance. Indeed, sources of such foliage are readily available in huge quantities, but are usually considered as mere waste. Indeed, other examples such as nettles are considered to be weeds, in the sense that they are generally unwanted but available in many environments in large quantities, especially on waste land, where the agent may ultimately be used. The same follows for seaweeds, which are also widely available and generally unwanted.

Therefore, large quantities of such plant material is available and is often wasted. As environmental concerns are increasingly important, it is an advantage of the present invention to utilise such waste, particularly in a method of remediation, which further improves the environment.

The terms charred material, carbon and charcoal are used interchangeably herein.

Without being bound by theory, the cations are absorbed to the carbon matrix of the charred material.

We have also surprisingly shown, in both woody and non-woody plants, that the ash/mineral content of the charcoal is related to the ability of said charcoal to adsorb cations. Thus, the ash content of the present charcoals correlates to the ability of said charcoals to adsorb pollutant metal ions, such as copper ions. It will be appreciated that the ash content and the mineral content of the charred material is linked and often the same.

Suitable ranges for the mineral contents of the present charcoals are provided below based on the proportion of ash (by weight) compared to the weight of the charcoal prior to extended heating (for instance 550 degrees C. for 12 hours). The charcoal may be prepared by charring at 450 degrees C. or less.

Preferably, the ash content is at least 15% (by weight of the charcoal), more preferably at least 15%, more preferably at least 16%, more preferably at least 17%, more preferably at least 17%, more preferably at least 18%, more preferably at least 19%, more preferably at least 20%, more preferably at least 22%, more preferably at least 25%, more preferably at least 30%, more preferably at least 35%, more preferably at least 40%, more preferably at least 45% and most preferably at least 50% or even 55%. Nettles and beets, being particularly preferred, have ash contents of between 40 and 50%.

Whereas ash content of the charcoals of this invention is a good indication of the charcoal's adsorbing capacity, it has to be appreciated that specific minerals within the charcoal are exchanged for metal ions. These minerals include potassium, magnesium, manganese and calcium. Some plants, such as horsetail, contain large amounts of silicate which is part of their ash content. Silicate is not exchanged for metal ions and does not contribute to the metal adsorbing properties of these charcoals. Similarly, halophytes and seaweeds contain large quantities of sodium salts to maintain cell turgor. This sodium contributes substantially to the ash contents of these plants, but is not exchanged for metal ions when the plants are charred.

Preferably, the plant material is capable of adsorbing large amounts of cations. Suitable reference cations are copper ions (Cu²⁺). Thus, it has been found that the weight of copper ions adsorbed by these materials is half to a third of the weight of the minerals that are contained in the charcoal. Thus, it is preferred that the weight of the minerals in the charcoal=2 to 3 times the weight of the adsorbed copper. In the case of charcoals that contain a large proportion of sodium or silicate adsorption is proportionally less. Adsorption of copper ions (by weight) equates to at least half the mineral content of the material, as calculated above, for instance. More preferably, this is a third, more preferably, this is at quarter or a fifth.

An even more precise prediction of the metal adsorbing abilities of the charcoals described here is provided by calculating the charge that is contained within the exchangeable minerals (K, Ca, Mg, Mn) that are present within the charcoal. Potassium has one unit of charge, while Ca, Mg and Mn all have two units of charge. By measuring the amounts of each of these minerals in the charcoal the charge contained on them can be expressed as ‘cmol charge’. This charge can be exchanged for an equal amount of charge present on the ions that are to be adsorbed (expressed as cmol). In a simple formula adsorption of metals can be expressed as: cmol metal/valency=cmol K+cmol Mg/2+cmol Ca/2+cmol Mn/2. It will be.appreciated that the ratio between the two sides of this equation is theoretically 1 but in practice not all the K, Mg, Ca and Mn will be exchanged, making the ratio >1. Furthermore, in solutions, potassium (in particular) is also exchanged for hydrogen ions, which further explains that the ratio between exchanged ions and metal adsorption is >1.

Furthermore, the present inventors have also found that the present charcoals are capable of raising the pH of a solution. In particularly preferred embodiments, the charred material when mixed with distilled, double distilled, deionised, demineralised or RO (Reverse Osmosis) water, in appropriate quantities, for example 0.5 g per 100 ml, the pH of the suspension is buffered to a pH of at least 10.0, more preferably to at least 10.1, more preferably at least to 10.2, more preferably to at least 10.3, more preferably to at least 10.35, more preferably to at least 10.4, more preferably to at least 10.45, more preferably to at least 10.5, more preferably to at least 10.55 and most preferably to at least 10.6 or above.

Suitable conditions for the pH buffering effect are described in the Examples. The pH may be measured based on, for instance, 0.5 g of finely grounded charcoal suspended in 100 ml demineralised water, the charcoal being kept in suspension and the pH measured after equilibrium has been reached.

In some embodiments, it is preferred that the charcoal is processed, for instance into a particulate or particulated form.

It will be appreciated that an ion exchange agent is an agent that is capable of or suitable for use in a method remediating selected environments that contain levels of cations, particularly metal ions, that is desired to be removed from said environment. This is particularly preferred where cations are toxic or harmful, especially ammonium, in bedding or clothing, or heavy metal ions in soil or solutions, by way of example.

The selected environment may be a brown-field site, such as the site of an old factory, mine or gasworks, for instance, where high levels of certain cations are often present in the soil, for instance. Thus, one particularly preferred embodiment is an ion exchange agent suitable for administration to soil. The agent may be mixed with the soil and either removed or, more preferably, retained in the soil. Indeed, it is one of the advantages of the present invention that the charred material may be left indefinitely in the environment, as the cations will be retained and bound within the charcoal and, therefore, their pollutant capacity is significantly reduced.

Suitable cations include organic cations, such as ammonium (NH₄⁺), as well as heavy metal cations such as copper, zinc, lead, mercury, nickel, aluminium and/or cadmium. The environment or area for treatment may be solid, liquid or gas, but is preferably soil or an aqueous waste, such as waste water or sewage, for instance.

Indeed, the present application has a number of applications that relate not only to the removal of metal ions, but also other organic cations, such as ammonium, as mentioned above. Particularly preferred applications of the present invention include adsorption of cationic dyes, for instance from waste streams; raising the pH of an environment, such as soil, to thereby precipitate the heavy metal ions.

Thus, the present invention also provides a method of removing a cationic dye from a solution, such as a waste stream, comprising contacting the present agent with said solution. Preferably, the agent is provided in the form of a filter or bed across which the solution flows.

The invention also provides a filter, preferably for a liquid or gas, comprising the agent. In a particularly preferred embodiment, the agent may be used in a water filter, preferably comprising polyurethane foam into which the agent is incorporated. In another preferred embodiment, the agent may be used in an air filter, for removing gaseous or gas-borne cations. These include mercury, which is often found in crematoria (derived from human fillings in human teeth). Metal smelters, power stations and incinerators, also tends to require air filters to remove metal ions from the air.

The agent may also be used in an apparatus for controlling the mineral content of a solution, preferably water and particularly for producing drinking or “mineral water.”

Also provided is animal bedding comprising the agent, which preferably may be admixed with straw or wood shavings, for instance. The agent in this instance must have been undergone substitution of the ions present on the charcoal with hydrogen ions, as described further below in reference to the acidified charred material.

The invention is also useful in composting as an enhancer or accelerator therefor. Means for altering levels of the cations in an environment are envisaged, comprising the present agent. These may include cosmetic products, such as face masks.

The agent is also useful as a means of retaining minerals in the soil, which would otherwise be lost by leaching. Thus, also provided is soil mixed with the agent, which may be applied to a susceptible area. The mixture may be provided with additional ions of which the plants in the area to be treated may be in need, such as sources of nitrogen, for example ammonium. Without further treatment, the charcoals of this invention are capable of supplying plants with important plant nutrients, which may, preferably, include potassium, calcium, magnesium and manganese. Indeed, the present invention provides a fertiliser comprising the present agent.

In a further aspect, the invention provides a plant growth medium comprising the present agent. Preferably, the medium further comprises fertilisers and/or seeds or plants for growing in said environment.

Preferably, the plant material is from fast growing plants or algae (such as macro algae), including seaweeds. Particularly preferred species of macro algae are bladder wrack (Fucus spp), oar weeds/kelp (Laminaria spp), thongweed (Hinanthalia spp) and sea lettuce (Ulva spp)

In a still further aspect, the invention provides a method where living plant material containing non-exchangeable ions is charred, thereby providing an ion-exchange agent.

The prior art (including JP2004035288A, CN1480396A, HU53581A, JP63159213A, JP05301704A and WO 96/29378A) largely focuses on methods of producing activated carbon from plant material. However, we focus on non-activated charred material that has ion-exchange properties and the useful commercial applications that arise from this, particularly in remediation of polluted environments or areas. Contrary to the teachings of the art, the charred material of the invention is not activated.

JP2006045003A discloses Cellolignin activated carbons. Although it does suggest deodorising properties of the carbon, the emphasis is on the need for mechanical and thermal treatment before steam activation of the charcoal.

JP2001252558A discloses the production of charcoal from general marine and agricultural waste, for use as a fertiliser. The charcoal can be made to absorb an aqueous sulphate solution with the purpose of adding a metallic ion. However, the metal ion is one that will be released into the environment for uptake by the plant. This is, we have found, likely to produce poor results. Indeed, the present invention is focused on adsorbing, i.e. taking up ions, in particular to remove toxic heavy metals from an environment to be treated (such as soil or water), which is in contrast to the release of ions as a slow release fertiliser taught in JP 2001252558. Furthermore, the method outlined in JP 2001252558 does not require that the metals are adsorbed to the carbon matrix, as simply mixing the charred material with the metals is sufficient with the carbon acting as a ‘bulking’ agent.

JP2001252558A also mentions the de-odorising effect on ammonia (i.e. it reduces the smell thereof), but teaches that the sulphate reacts with the ammonia to provide ammonium sulphate, which is a useful fertiliser.

CN1944246A focuses on the need to overcome a lack or raw materials for charcoal and discloses material is derived from roots from 3 year old Chinese “giant reeds” as the solution. It goes on to teach that the charred material should be activated at high temperatures. The uses of the activated charred root material can include removing heavy metals, but this is expected as all charcoals have some, albeit limited, ability to adsorb such ions. In contrast, we have found that living plant material, especially young foliage, when charred but not activated, shows excellent metal ion adsorbent properties, due to the mineral content of the source material.

The charring process is well known to those skilled in the art. Essentially, it involves heating to temperatures considerably above boiling (for instance between 400° C. and 700° C.), under oxygen starved conditions. Temperatures much above this level can cause unwanted degradation even in the absence of oxygen. Thus, the absence of an oxidizing agent, such as an acid, steam or air is particularly preferred. The temperature will normally be selected according to the substance to be charred and the extent to which it is desired to drive off unwanted organic substances. The process does not normally need to be air-tight, as the heated material generally gives off gas, but circulation of atmospheric air should be avoided as much as possible. The aim is to maximise char production and maintain a high mineral content within the charcoal.

This can be achieved via a number of techniques including slow pyrolysis at temperatures of between 300 and 500° C. The yield of products from pyrolysis varies heavily with temperature. The lower the temperature, the more char is created per unit biomass. High temperature pyrolysis is also known as gasification, and produces primarily syngas from the biomass. The two main methods of pyrolysis are “fast” pyrolysis and “slow” pyrolysis. Fast pyrolysis yields 60% bio-oil, 20% biochar, and 20% syngas, and can be done in seconds, whereas slow pyrolysis can be optimized to produce substantially more char (-50%), but takes on the order of hours to complete. Both methods will yield suitable charred material according to the invention.

When a small quantity of charcoal (say 1 g) is mixed with a large volume of water (say 1 litre) the pH of the resulting suspension will rise dramatically, often well above pH 10 as a result of the removal of positively charged hydrogen ions from the water. Alternatively, if a small amount of the charcoal (say 1 g) of this invention is mixed into a litre of acidic solution with a pH of 2 or 3, the charcoal will quickly neutralise the solution to a pH of 7 or 8. This is a particularly useful aspect of this invention for the removal of toxic metals from the environment because the charcoals not only will adsorb dissolved metal ions but will also cause their precipitation in the form of metal salts (often on the charcoal surface itself where the pH is highest). In this respect, charcoals of this invention can be used to replace ‘liming’ of agricultural soils to remove acidity.

The invention also provides an agent used for composting of organic waste, such as garden waste, manure or sewage. During composting a variety of cations are released including ammonium ions. Such cations are normally highly mobile and are easily lost from the system. By mixing the agent into the waste before the composting starts, a compost can be created that retains more nutrients while any toxic metals that are present in the material are stably bound onto the charcoal, making them non-toxic. Composting is just given here as an example and it should be appreciated that mixing charcoal of this invention to any degradable organic source could be beneficial. For example, mixing the charcoal of this invention with poultry litter will result in the binding of ammonium that is generated when the uric acid that is present in the bird faeces is converted to ammonium ions.

Substances used to produce the charcoal of the invention are normally chosen from fast growing plant shoots and leaves or macro-algae. Suitable materials are, preferably, young wood, young bark as well as leaves. Many woody and non-woody plants and algal (both micro-algal and macro-algal) species are suitable, and are discussed below, but those that are high yielding, and are easy to grow are most preferred. Stinging nettle, dead nettle, beet (sugar beet, sea beet and chard for example), crucifers (cabbage, oilseed rape) and spinach are examples. When woody plants are used it are the young branches and leaves of rapid growing trees such as eucalyptus, poplar, and willow that are most suitable.

In an alternative aspect, the present invention provides a charcoal prepared from plant leaves and stems. In particular, straw from crops, for instance oilseed rape, is highly effective as a source materials for the charcoal of the present invention.

The present invention further provides a charcoal prepared from one or more polyol phosphates. Polyols are carbon chain molecules bearing a plurality of hydroxyl groups. Suitable examples include glycerol (propane-1,2,3-triol), maltitol, sorbitol, and isomalt.

The present invention further provides the use of charcoal as described herein in removing or binding cationic species in an area. The cationic species is preferably one or more metal species whose bio-available concentration it is desired to reduce, such as copper, zinc, lead, mercury, nickel and/or cadmium. The area may be solid, liquid or gas, but preferably is soil or an aqueous waste.

Charcoal of the invention, when prepared from non-woody materials, will often be friable or in powder form. Accordingly, treatment of the area may be by trapping the charcoal in a vehicle and passing a liquid over or through the vehicle, thereby to contact the trapped charcoal and permit removal of some or all of the contaminating cations. To allow more easy passage through the charcoal thus entrapped, the charcoal can be mixed with coarser materials including wood charcoal, or coarse sand or gravel. The liquid may be the form of the area to be treated, or a slurry with, for example, water may be formed. The charcoal may be used without a vehicle where it is acceptable to leave spent or partially spent charcoal as a component of the area to be treated. If a vehicle is used, it is advantageously selected so as to permit removal from the area and/or to support other treatment means, such as an arsenate chelator or microbes.

Suitable vehicles may be any porous matrix able to retain the charcoal. In this respect, thermoplastic materials, or natural polymers, such as cellulose, can be annealed to adhere charcoal powder for example, or the charcoal may be mixed with a foam that sets, retaining the charcoal.

Where the area is soil, the charcoal may be used on its own, in a vehicle, as described, and/or together with other treatments.

The invention further provides a method for treating an area comprising contacting the area with the agent as described, and subsequently removing the charcoal if desired. Removal, especially when incorporated into polluted soil and slurries, is often not necessary, as the presence of the charcoal can help to stabilise the material, and we have shown that, for example, acidic soils can be at least partially neutralised using the charcoals of the invention.

Thus, in a further aspect, there is provided the use of a charcoal as described to raise the apparent pH of acidic soil toward pH 7 or higher by contacting the soil with the charcoal in an amount and for a period sufficient to elevate the pH of the soil.

Charcoals derived from stinging nettle, dead nettle, beets, bladder-wrack, and a range of other similar materials are particularly preferred.

Charcoals made from stinging nettle (Urtica dioica) and white dead nettle (Lamium album) and beets; for example, outperform synthetic zeolites by a factor of 3.77 and natural zeolites by a factor of 32 in terms of Cu²⁺ adsorption. For Cd ions, charcoals derived from stinging nettle adsorbed 1.78 mmol Cd/g charcoal, which is 4× greater than the adsorption of Cd onto synthetic zeolites and 43× greater than adsorption Cd onto natural zeolites. Thus, charcoals derived from stinging nettle and dead nettle were found to adsorb 18-20% of their weight in Cd and Cu and up to 30% of their weight in Hg. For Zn this percentage was 12%, equivalent to 1.85 mmol Zn/g charcoal, which is 2.5× better than adsorption onto synthetic zeolites and 35× better than adsorption onto natural zeolites.

Examples of other materials useful in the present invention include; charred brassicae (plant species of the cabbage family), charred oilseed rape, charred wheat straw, charred bracken, charred horsetail, and charred seaweed [for example: bladderwrack (Fucus vesiculosus)], each being capable of adsorbing >1 mmol Cu/g charcoal and, therefore, superior in their adsorbing potential than even the best performing synthetic zeolites.

Particularly preferred are beets and family members thereof, with sugar beet being particularly preferred.

Because the charcoal of the present invention raises the pH of the environment considerably, adsorption will occur from an acidic environment once the pH of that environment has been neutralised to a pH of 4.5 or more. This buffering effect on pH has the advantage that no toxicity occurs by desorption of adsorbed metals in situations where the polluted environment may be subjected to an input of acidic materials such as acid rain. In fact, when applied to an already acidic environment, the charcoals of the invention can remove metals effectively from solutions that have a pH as low as 3 by raising the pH toward neutrality, as is shown in the accompanying Examples. In contrast, zeolites do nothing to ameliorate low pH areas.

The adsorbent properties of the charcoal derived from plant materials can be dramatically improved by the careful selection of the growth conditions of the plants. For example, stinging nettles growing under oligotrophic conditions on a chalk rich hill side produced charcoal with a maximum absorbance of 60,000 ppm Cu (0.94 mmol/g) while charcoal derived from stinging nettles that grew on a nutrient rich manure heap adsorbed 200,000 ppm Cu (3.13 mmol/g—c.f. accompanying Examples).

Thus, instead of altering the adsorbent properties of charcoal using activation procedures that can be time-consuming and expensive, it is now possible to select the properties of the charcoal by growing plants under conditions selected to optimise the adsorbent properties of the charcoal produced therefrom.

Within plant species suitable for use in the present invention, preferred plants are those with dark green foliage. Both the plant species and the colour of the leaves, as a reflection of the nutritional circumstances of the plant, are important. Thus, this phenotypic selection will favour, to some extent, plants capable of extracting high levels of mineral nutrients from soils and which are therefore capable of fast growth.

After selection of a suitable plant species, darker green plant material typically gives rise to highly adsorbent charcoals, while charcoal produced from small plants with yellowish foliage are generally less adsorbent. Thus, selection of plants by phenotype is a useful guide to which plants yield the most advantageous charcoal of the invention. In addition, it is typically the green part of the plant that has the best properties, especially leaves and young stems. This is a particular advantage, as the woody portions of the plant may then be used for other purposes or other types of charcoal, leaving the leafier parts, which might otherwise have gone to scrap, to be used in accordance with the present invention.

The charcoals of the present invention are microbially inert (non-degradable) and once metals are bound onto the charcoal the binding is stable, making application to soil a long term option. Charcoal of the present invention added to soil can be used to permanently break metal—receptor linkages, resulting in metal contaminated soil becoming non-toxic after charcoal application.

Nettles are a common weed and the cultivation of nettles has already been practised, such as for the production of fibres to produce nettle cloth. For farmers already growing nettles, the present invention is useful, as the waste material, which is mainly leaves, is typically the best for manufacturing the charcoal of the invention. Without being restricted by theory, two or three crops/year are generally possible, and a yield of >2 tonnes of nettle charcoal per hectare may be obtained.

More advantageous however is the use of agricultural waste materials or by-products that have currently no or little economical value, such as sugar beet tops and oilseed rape straw. Especially sugar beet tops when charred produce a charcoal that is highly adsorbent and the tops are easy to collect.

In experiments to establish whether soil contaminated with heavy metals could be remediated, charcoal derived from stinging nettle was used to treat mine tailings containing more than 1600 ppm Cu, and more than 800 ppm Cd. After application of 5% (v/v) charcoal (equivalent to 0.4% charcoal by weight) an almost complete immobilisation of bioavailable metals was found, which resulted in a restoration of plant growth and microbial activity. Higher application rates gave generally better and longer lasting results (c.f. accompanying Examples).

Charcoals derived from herbaceous plants and seaweeds are, in general, less robust than charcoals derived from woody materials. Thus, these charcoals can readily be made into a slurry that can be directly applied into contaminated soil, such as by injection. It will be appreciated that, in case of severe compaction, the soil should be first advantageously loosened to create space for the charcoal suspension. In this way the charcoal can disperse via cracks and fissures in the soil. Since metals normally would disperse through soil in the aqueous solution, such an application would effectively remove these mobile metal ions.

To avoid the possibility of fine particles clogging together in effluent streams, thus impeding water flow, charcoals of the present invention may conveniently be embedded in a porous material, so as to allow contact of dissolved metals with the charcoal. Such a porous material is ideally strong and/or hydrophilic, preferably both. Suitable materials include polyurethane foams and natural polymers, such as cellulose, that can be made into sponge-like materials. These materials may be made to selected specifications to increase strength, hydrophilic properties and porosity. It will be appreciated that polyurethane and cellulose are simply two examples of useful carriers for charcoal particles, and that other porous polymers are possible.

Using granules made of polymer, or other binding materials, such as cement, that hold the charcoal allows application to systems where free flow is essential. Furthermore, formulation of the charcoal into a granule made of polymer allows for the carbon to be combined with other treatment systems that complement the ability of charcoal to adsorb cationic metal species.

The charcoals of the present invention bind cations well. Their ability to bind anions, such as arsenite [As(III)] and Arsenate [As(V)], is not good, and the charcoals of the present invention also tend to increase the pH of the soil, so that arsenic is rendered more soluble. Co-application of iron-oxide, such as in granules or separately, binds free arsenic anions. In a preferred, granular formulation, metal adsorbent charcoals of the present invention are combined with charcoals or other substances suitable to bind organic pollutants.

We have also shown that potassium is one of the main exchangeable element of charred material or charcoals made from nettle, beet and so forth. When brought into the environment, potassium is also exchanged with hydrogen ions. However, where it is desired to keep the pH low or stable, this uptake of H+ ions can be disadvantageous.

Accordingly, it is preferred that the charred material of the present invention has less than 50% of its natural K ions, the K ions having been replaced by other metal ions, preferably Mg or Mn and most preferably by Ca. ions. Preferably, at least 60%, more preferably at least 70%, more preferably at least 80%, and most preferably at least 90% of the charred material's natural K ions are exchanged to provide said modified charred material.

The natural K ions are those present in the charred material prior to modification. This may be achieved by contacting the present charred material with a source of Ca ions, most preferably an aqueous solution of a Ca salt, preferably Calcium Chloride. The modified charred material, preferably derived from nettles, is preferably capable of adsorbing more than 200,000 ppm of Cu ions from a Cu solution as herein described, more preferably at least 220,000 ppm, more preferably at least 240,000 ppm, more preferably at least 250,000 ppm and most preferably at least 270,000 ppm of Cu ions from a Cu solution. Similar results would be expected with Nickel. Preferably, the modified charcoal has a greater capacity to adsorb metal ions as displacement of potent ion binding sites with hydrogen ions is limited. Therefore, thus modified charcoals preferably adsorb up to 25%, and more preferably up to 50%, more Cu ions from solution than non-modified ones.

Preferably, the charred material does not change the pH of normal tap water by more than 1.5 pH units, and preferably by 1.0 units or less when 0.5 g of the charcoal is mixed with 100 ml water, preferably tap water.

A cheap ion-exchange material that releases hydrogen ions to lower the pH of the medium could be advantageous in media such as animal beddings, where a low pH would prevent the conversion of ammonium to ammonia. The advantage of using acidified charcoals is that these materials are long-lasting and are less reactive under moist conditions than acidic salts such as alum and hydrogen-bisulphate. We have surprisingly found that acidified non-activated charcoal lowers the pH, thus preventing the formation of ammonia. Without being bound by theory, to date we have not found that ammonium is adsorbed with these materials

The acidified charred material is preferably obtained by grinding charred material, most preferably from nettles or other materials described here, and treating this with an acid. The acid can be a weak acid or a strong acid, such as hydrochloric or nitric acid, provided that the acid is at least pH 3 or 4 or lower. The acid is preferably at least 0.5 molar and more preferably at least 1M or more. Preferably, the mixture is left until at least 70% and more preferably at least 90% of the acid was removed from solution by the charcoal, such that the pH of the solution has a pH of 3 or less, more preferably pH 2 or less and most preferably pH 1 or less. The resulting acidified charred material is drained and subsequently dried and has a pH of around 4 when added to water.

Thus, the invention provides an ion exchange agent as defined herein, modified after charring, wherein naturally occurring Potassium ions are replaced by other suitable cations, which may include metal ions such as Calcium, Manganese or Magnesium, or Hydrogen ions.

The agent is preferably acidified non-activated charred material having a pH of around 4 when added to water or a solid matrix such as soil or animal bedding. The acidified charred material is capable of acting as weak acid itself and can be used to modify or buffer its environment by releasing H ions and, advantageously, adsorbing other cations to replace the lost H ions.

Also provided is a method of providing said acidified charred material as discussed above, wherein metal cations such as K or Ca ions, naturally in charred material prior to acidification, are replaced by the H ions.

The acidified charred material is especially useful in animal bedding, so the invention provides animal bedding, particularly that described above, comprising the same, preferably comprising a mixture of the animal bedding (for instance straw, wood chippings, saw dust or cat litter) with the acidified charred material. Preferably, the present acidification occurs at ambient temperature (around 25 degrees C.).

Although the addition of strong acids to charcoal is known, this is to create activated charcoal and thus increase the surface area of the charcoal, which is not required in the present acidified charred material. Activation is achieved at high temperature and in the presence of an oxidising agent, i.e. the strong acid or an oxidising gas, such as steam or air. Such conditions are thus disclaimed. In fact, the present acidified charred material is not activated as it is disadvantageous to increase the surface area of the acidified charred material that could also lead to the loss of minerals in the charcoal which results in poor metal adsorption.

Preferably, the acid used to provide the acidified charred material is either a weak or a strong acid. It is also preferred that the temperature is ambient or lower than that used in activation processes.

The invention will now be described with reference to the following non-limiting Examples.

### EXAMPLES

### Example 1

Metal Adsorption onto Nettle Charcoal Compared to Metal Adsorption onto Charcoals Rich in Phosphate

Methodology

To test the significance of phosphate groups for metal adsorption, three different materials were used for charring. Glycerol phosphate and bone meal are both high in P, while stinging nettle contains relatively little P (ca. 10% of the P in either bone or glycerol phosphate charcoal) (Table 1). Metal sorption to their charcoals was quantified using AA (Atomic Adsorption).

TABLE 1
Total and water soluble phosphate levels for glycerol
phosphate, bone and nettle charcoals.

| | Total Phosphate | Water Soluble Phosphate |
|---|---|---|
| | (mg P/kg) | (mg P/kg) |
| Glycerol Phosphate | 195694 ± 16532 | 2547 ± 80 |
| Charcoal | | |
| Bone Charcoal | 120133 ± 3401 | 220 ± 9 |
| Nettle Charcoal | 15590 ± 2639 | 96 ± 49 |

Values are shown as mean ± standard error of the mean. N = 3.

Results

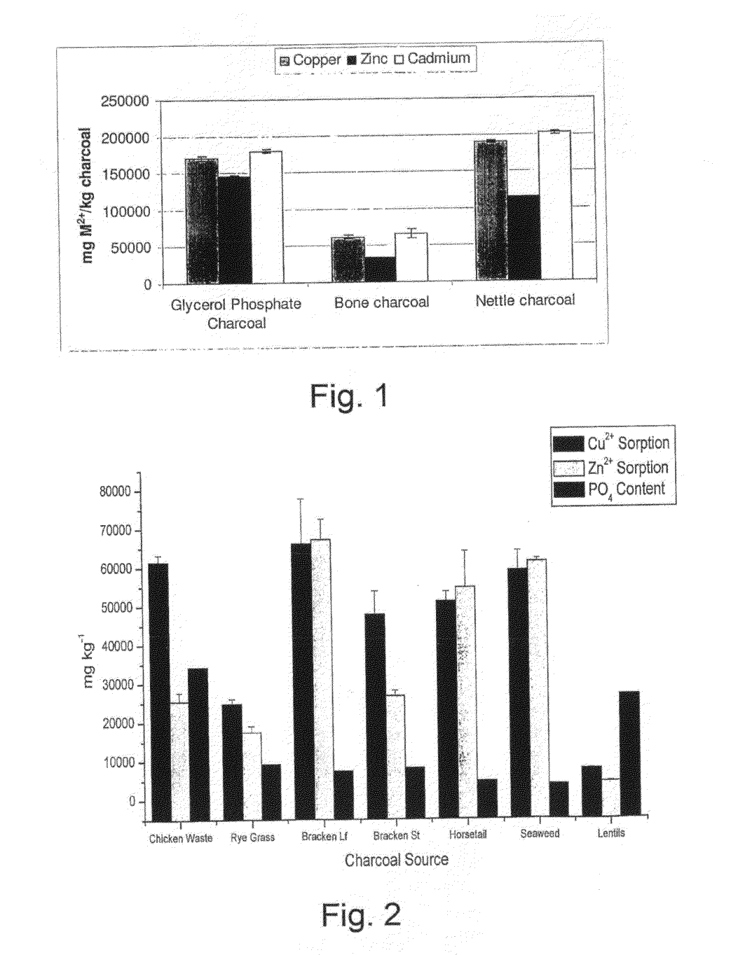

Glycerol phosphate charcoal and nettle charcoal adsorbed around three times more of all three metals than bone charcoal. Results are shown in FIG. 1, wherein P<0.001 and results are shown as mean±standard error of the mean. N=3. Nettle charcoal adsorbed slightly more copper and cadmium but significantly less zinc (P<0.001) than glycerol phosphate charcoal. All three charcoals adsorbed metals ions in the order Cd>Cu>Zn.

Conclusions

Nettle charcoal contains only 10% of the P present in either bone charcoal or glycerol phosphate charcoal, but its ability to adsorb metals was as high, or higher, than that of either of the P rich charcoals, suggesting that metal adsorption in nettle charcoal is not solely determined by phosphate groups.

### Example 2

Adsorbing Properties of Charcoals Derived from Different Plant Materials

Methodology

A range of organic materials was selected, some of which were known to be high in P, such as chicken litter and lentils. For others, P content was unknown, but presumed to be lower than either chicken litter or lentil seed. All materials were charred at 450° C. and the resulting charcoals were tested for their ability adsorb Cu. P content of each charcoal was quantified to determine whether there was any correlation between P content and metal adsorbing properties of the charcoals.

The results are shown in FIG. 2. N=3.

Conclusions

Charcoals derived from non-woody materials such as seaweed (bladder-wrack), horsetail, and bracken, adsorb large amounts of metal (up to 60,000 ppm Cu and Zn).

There is no correlation between P content and metal adsorption. Materials high in P, such as lentils, showed least metal adsorption, while charcoals derived from seaweed, horsetail, and bracken, had low P content but high metal adsorbing potential.

### Example 3

Precipitation of Metal Salts on Charcoal Surfaces

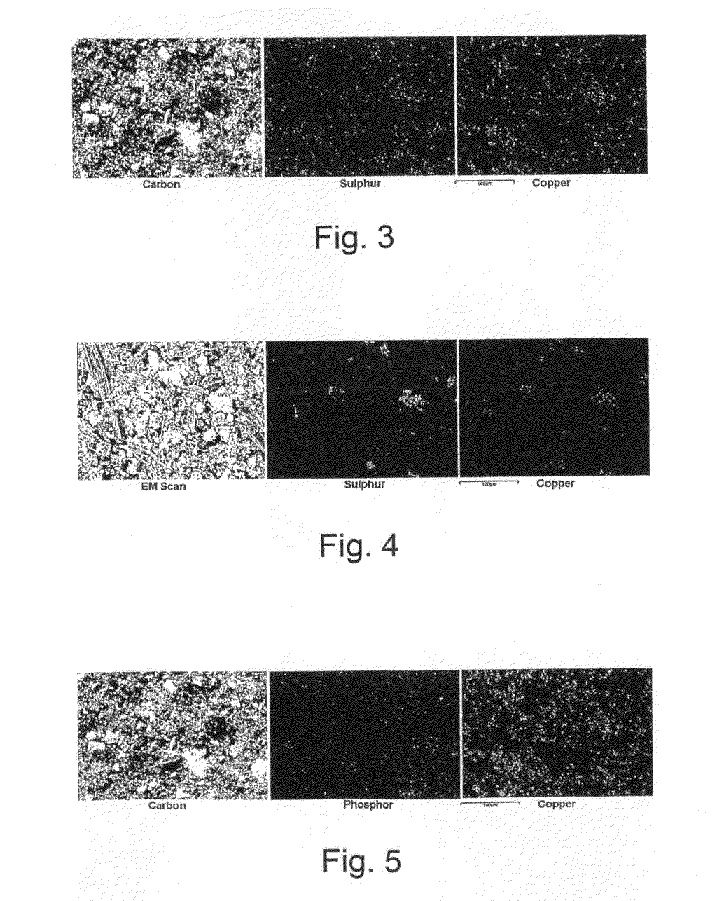

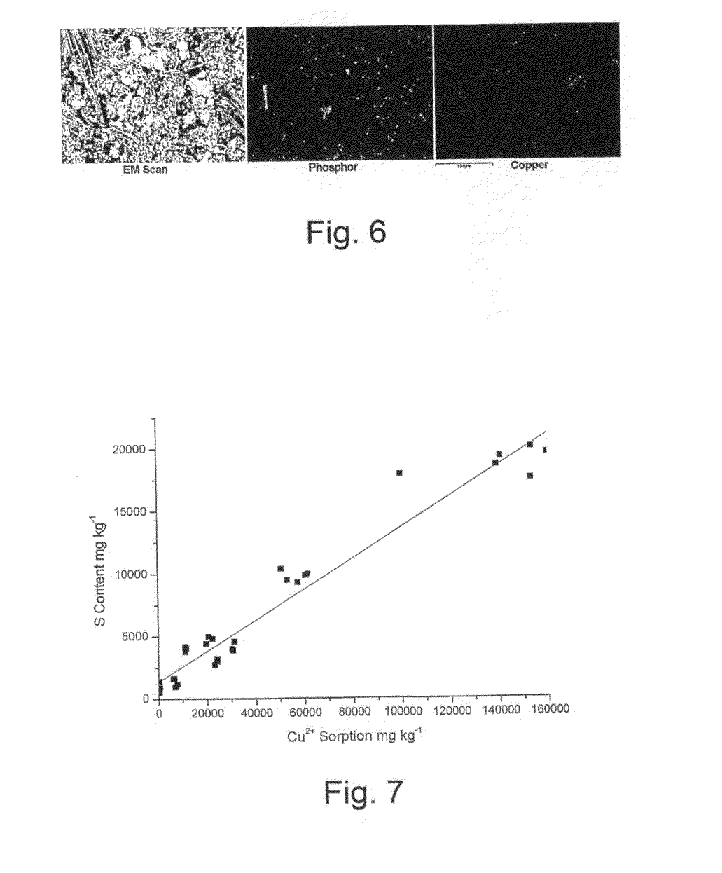

Solutions of CuSO₄ (250 ppm) were prepared and charcoal derived from bladder wrack and stinging nettle were added at a rate of 2 g/l. After shaking for 24 h the charcoal was filtered out and washed with RO water. EDX micrographs of the thus treated charcoal showed close matches between areas high in sulphur with areas high in copper on charcoal produced from bladder-wrack, and stinging nettle, while showing a poor match between areas high in phosphor with areas high in copper on charcoal produced from bladder-wrack, and from stinging nettle. The results are shown in FIGS. 3 to 6. FIG. 3 is an EDX micrograph showing a close match between areas high in sulphur with areas high in copper on charcoal produced from bladder-wrack (Fucus vesiculosus). FIG. 4 is an EDX micrograph showing a close match between areas high in sulphur with areas high in copper on charcoal produced from stinging nettle. FIG. 5 is an EDX micrograph showing a poor match between areas high in phosphor with areas high in copper on charcoal produced from bladderwrack (Fucus vesiculosus), and FIG. 6 is an EDX micrograph showing a poor match between areas high in phosphor with areas high in copper on charcoal produced from stinging nettle.

Conclusions

In charcoal derived from stinging nettle and bladderwrack, there was a good match between adsorbed copper and areas rich in sulphur, while there was no obvious match between adsorbed copper and phosphate groups. Whereas it is conceivable that sulphur groups present on the charcoal are responsible for metal binding, a more likely explanation is that as a result of the high pH created on the charcoal surface precipitation of CuSO₄ occurred.

### Example 4

Precipitation of Metal Salts on Charcoal Surfaces

To determine if there was a correlation between the metal adsorbing properties of charcoals derived from different source materials and the amount of metal salts that would precipitate on their surface.

Methodology

Besides stinging nettle, a range of plant materials were selected for their different metal sorption capacities including garlic, cabbage, stinging nettle, dead nettle, sweet chestnut bark, sweet chestnut wood (old), young sweet chestnut wood, bladderwrack, horsetail, lentils, pine wood and sewage cake. These materials were dried at 25° C. and charred at 450° C. and their metal adsorbing properties were compared against materials with low adsorbent properties [mature sweet chestnut wood (Castana sativa)] or plants that were similar to stinging nettle in appearance and habitat (dead nettle).

Samples were subsequently ground to a fine charcoal powder and 0.5 g of each was suspended in 250 ml Cu sulphate at a concentration of 250 ppm. After filtering and rinsing of the charcoal, each sample was ashed at 450° C. and digested using aqua regia. Copper in the resulting solution was analysed by Inductively Coupled Plasma Optical Emission Spectroscopy (ICP-OES). Sulphur content was determined externally by NRM Laboratories Ltd, UK. Three samples for each source material were used. Cu adsorption vs. sulphur content were subsequently plotted and a correlation coefficient calculated.

FIG. 7 shows the correlation between sulphur content and Cu²⁺ sorption capacities of several charcoals made from:—garlic, cabbage, stinging nettle, dead nettle, sweet chestnut bark, sweet chestnut wood (old), one year old sweet chestnut wood, horsetail, bladder wrack, pine wood, lentils and sewage cake. Charcoal particles were suspended for 48 hours in metal solutions containing Cu²⁺ at 250 mg l⁻¹. Three samples for each material were used.

Results/Conclusions

There was a very strong correlation between the ability of a charcoal to adsorb copper and sulphur content of that charcoal (r²=0.9572).

Precipitation of CuSO4 occurred according to the adsorbent properties of the charcoal. However precipitation of metal salts only accounted for 12% of the metal adsorption of the charcoals tested.

### Example 5

Adsorption of Metals from Acid Solutions

Methodology

To show how effective different charcoals are at removing metals from an acidified solution, finely ground bone, glycerol phosphate and nettle charcoals were suspended in acidified solutions containing 250 mg CuSO₄/l at a rate of 2 g charcoal/l. Charcoal was kept in suspension using an electric stirrer. Each flask contained excess Cu in relation to the amount of charcoal that could be adsorbed by the suspended charcoal. Solutions were acidified using HCl to pH 6, 5, 4, 3, 2 and 1. After 48 hours the charcoal was filtered out, rinsed and digested in concentrated nitric acid. The amount of Cu adsorbed was assessed using AA.

Results

The results are shown in FIG. 8, which shows adsorption of Cu²⁺ from solutions acidified to pH 4, 3, 2 or 1, by nettle charcoal and charcoal derived from glycerol phosphate. N=4.

Conclusions

Charcoal derived from stinging nettle was effective at removing metals from solutions with a pH of 3 by neutralising the pH of that solution.

Charcoal derived from glycerol phosphate was effective at removing metals from solutions with a pH of 2.

It should be noted here that the charcoal is thought to raise the pH of the solution as it appears that the metals are taken up at low pH, whilst in fact the solution is buffered to a pH of 4 or higher.

### Example 6

Restoring Plant Growth on Mine Tailing using Nettle Charcoal.

Methodology

Mining waste was collected from a tin mining spoil heap in the Tamar Valley area (Dartmoor, England). The material was passed through a 2 mm sieve before any analysis of available metals. Analysis of EDTA and DPTA extractable metals, as well as total metal content was undertaken by NRM Ltd. Selected physiochemical properties and micronutrient analysis of original soil are given in Table 3.

TABLE 3
Selected physiochemical properties and micronutrient
analysis of Tamar Valley soil.
Total Metals (dry weight mg kg⁻¹)

| | |
|---|---|
| Copper | 1641 |
| Zinc | 47.2 |
| Lead | 189 |
| Cadmium | 813 |
| Chromium | 33.8 |
| Arsenic | 34470 |

EDTA Extractable Metals (mg l⁻¹)

| | |
|---|---|
| Copper | 18.2 |
| Zinc | 0.8 |

DPTA Extractable Metals (mg l⁻¹)

| | |
|---|---|
| Iron | 274.6 |
| Manganese | 1.1 |

Cation Availability (mg l⁻¹)

| | |
|---|---|
| Phosphorous | 16.6 |
| Potassium | 29 |
| Magnesium | 12 |
| Soil pH | 3.2 |

To improve water holding ability of the material, the mining material was mixed to a ratio of 1:1 with perlite (diam. <2 mm) This mixture of spoil material and perlite is further referred to as ‘soil’. Soil pH was determined with a Hanna 250 pH meter using a 1:10 soil/water suspension. Viable microbial counts were made by mixing 1 g soil with 9 ml Ringer's solution and shaking to create a bacterial suspension. Bacterial suspensions were diluted and plated onto 1 Tryptone Soya Agar and plates were incubated at 20° C. for 7 days before plates were counted

Soil amendments used in this study were: stinging nettle charcoal (NetC) and sweet chestnut (Castana sativa) charcoal (SwChC). These were compared to controls that were amended with perlite only (Table 4). NetC was produced from mature stinging nettles (Urtica dioica). SwChC was produced from 2 year old stems harvested from a sweet chestnut coppice in the summer. All plant materials were air dried at 60° C. then charred at 450° C. using a Carbolite LMF 4 muffle furnace by wrapping the material in several layers of aluminium foil before heating. Charcoals were ground and sieved to <2 mm in size. Table 4 shows the different treatments that were compared.

TABLE 4
Different treatments to metal contaminated soil.

| Additions (w/w) | Soil % | Charcoal % | Perlite % |
|---|---|---|---|
| 4% Charcoal | 96 | 4.0 | 0.0 |
| 2.0% Charcoal | 96 | 2.0 | 2.0 |
| 1.0% Charcoal | 96 | 1.0 | 3.0 |
| 0.4% Charcoal | 96 | 0.4 | 3.6 |
| 0% Charcoal | 96 | 0.0 | 4.0 |

Soil consisted of 50% mining spoil (v/v) and 50% perlite (v/v). N = 3.

To assess bio-available metals in soil, a batch leaching experiment was used (Bsulphur EN 12457-2:2002), using all soil/charcoal combinations. In brief, a 20 g sample (dry weight) of soil was placed into a 250 ml conical flask. Flasks were set up in triplicate for each soil/charcoal combination. To each mixture 180 ml of deionised water was added that had been left exposed to the air overnight to allow CO₂ to dissolve. Flasks were sealed and shaken at 200 rpm for 24 hours. After shaking, samples were allowed to settle for 20 mins after which the supernatant was drawn off and suction-filtered through a Whatman filter paper number 1. The solution was analysed by Atomic Adsorption (AA) for copper, zinc and arsenic.

Results

Effect of Charcoal Amendments on Metal Leaching

Immediately after amendment with as little as 0.2% (w/w) nettle charcoal reduced the amount of leachable Cu by 80% and larger quantities removed all leachable Cu (FIG. 9). In contrast sweet chestnut (Castana sativa) charcoal was relatively ineffective at reducing the amount of leachable Cu immediately after amendment with charcoal (FIG. 9). Adding as much a 4% sweet chestnut (Castana sativa) charcoal by weight reduced the leachable Cu by <50% (FIG. 9). FIG. 9 shows leachable copper (mg Cu/kg soil) in soil amended with charcoal derived from stinging nettle or sweet chestnut 24h after amendment (n=3).

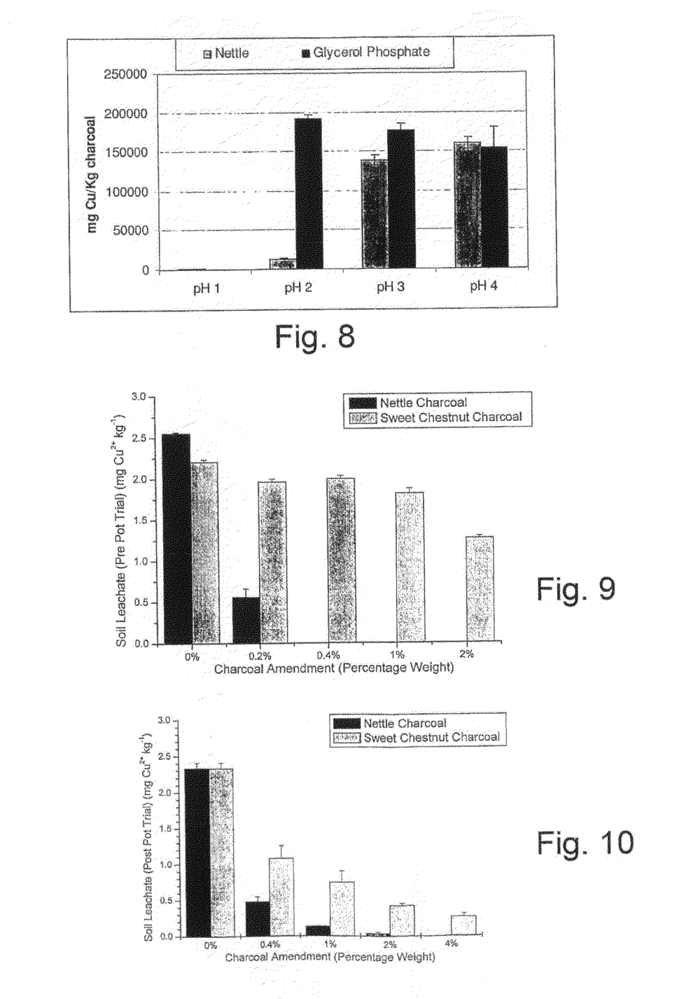

Fifty five days after amendment with charcoal derived from stinging nettles, effective (>99%) adsorption of leachable Cu was achieved with amendment rates >2% by weight. Sweet chestnut (Castana sativa) charcoal reduced the amount of leachable Cu was reduced by >80% when >2% (by weight) charcoal was added (FIG. 10). FIG. 10 shows leachable copper (mg Cu/kg soil) in soil amended with charcoal derived from stinging nettle or sweet chestnut, 55 days after amendment and after the soil was used to support plant growth (n=3).

Conclusion

Nettle charcoal effectively immobilises leachable metals in soil.

### Example 7

Effect of Charcoal Amendments on Soil pH

Addition of as little as 0.4% nettle charcoal to soil significantly increases soil pH (ANOVA all vs. control p<0.01). Further increases in nettle charcoal amendment continue to raise soil pH. At 2% amendment the soil pH reached neutrality (2%: pH=6.78, 4%: pH=6.83). Results are shown in FIG. 11, which shows soil pH after a 40 day pot trial growing sunflowers in soil amended with different concentrations of nettle and sweet chestnut charcoal. N=3. Error bars show standard error.

It can be seen from FIG. 11 that addition of sweet chestnut charcoal significantly raises the soil pH only at the maximum amendment of 4% where the pH is increased to 5.54 (P<0.01).

Conclusion

Charcoals produced from stinging nettle are better at raising soil pH than those produced from sweet chestnut wood.

### Example 8

Effect Of Charcoal Amendments On Plant Growth—Stem Height

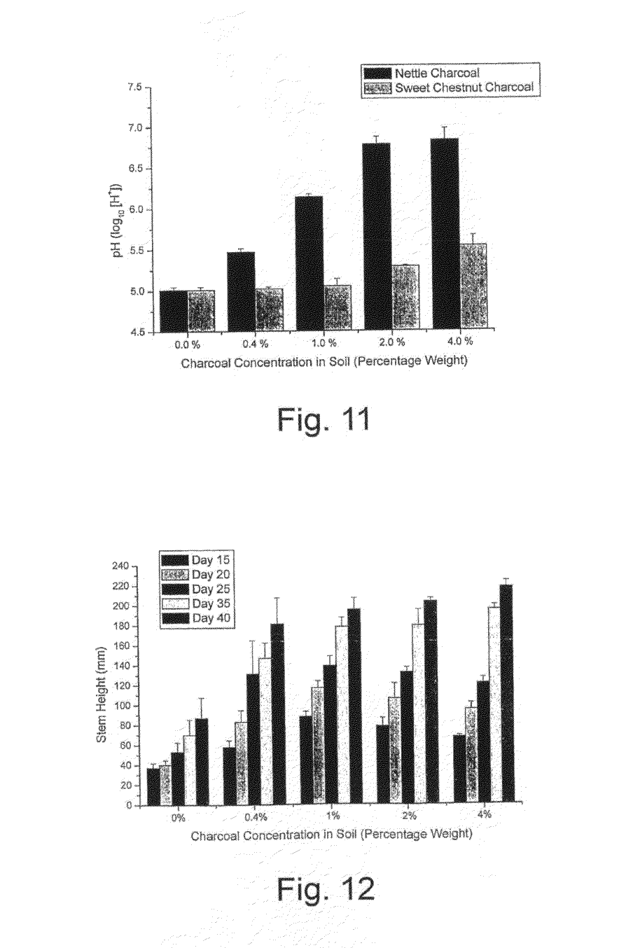

Addition of as little as 0.4% nettle charcoal to soil, significantly increases stem height after 15 days (p<0.05). After 40 days pots with nettle charcoal amendments produced plants that were between 2 and 2.5 times higher than those of the non-amended control. There were no significant differences between plants grown in soil with 0.4, 1, 2 and 4% nettle charcoal amendments after 40 days (p>0.05). (FIG. 12) Addition of 0.4% sweet chestnut (Castana sativa) charcoal to soil significantly increases stem height after 20 days (p<0.05). Pots with 2% sweet chestnut (Castana sativa) charcoal produce significantly increased stem heights after only 15 days (p<0.05). FIG. 12 shows sunflower stem height over time of plants growing in soil with different concentrations of nettle charcoal. N=3. Error bars show standard error.

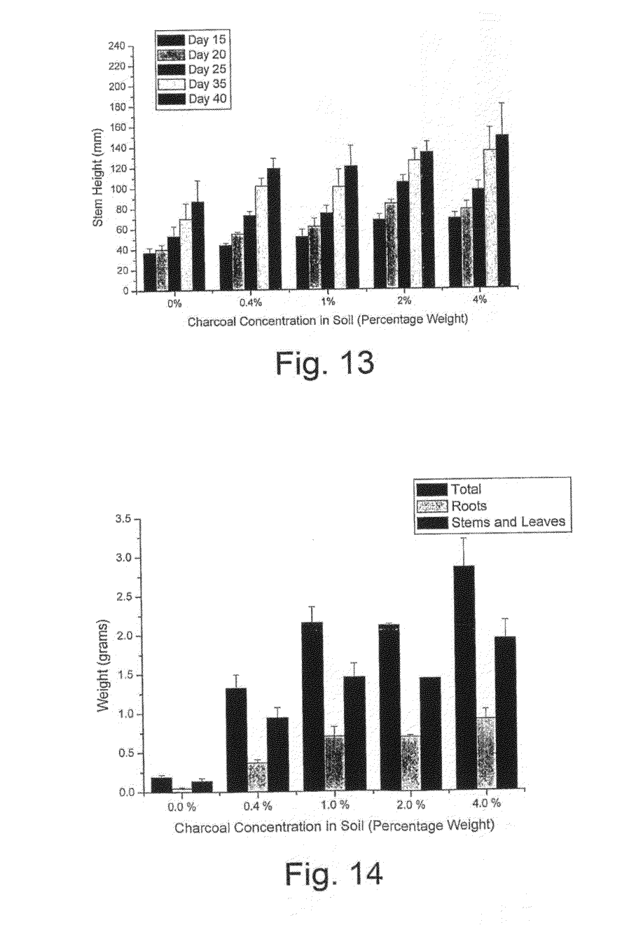

After 40 days, pots with sweet chestnut charcoal amendments produce plants between 1.3 and 1.7 times higher than those of the controls. There are no significant differences between pots with 0.4, 1, 2 and 4% sweet chestnut charcoal amendments after 40 days. FIG. 13 shows sunflower stem height over time of plants growing in soil with different concentrations of sweet chestnut charcoal. N=3. Error bars show standard error.

### Example 9

Effect of Charcoal Amendments on Plant Growth—Biomass

All nettle charcoal additions produce significantly increased, root biomass and stem and leaf biomass dry weights after 40 days growth (P<0.01). Addition of 4% nettle charcoal compared with 0.4% results in plants with significantly increased biomass (P<0.01). Comparisons of other additions excluding the control produce non-significant differences (P>0.05).

After 40 days, pots with nettle charcoal amendments produce plants that were between 8 and 20× heavier than those of the control. FIG. 14 shows sunflower dry biomass after 40 days growth in soil with different concentrations of nettle charcoal. N=3. Error bars show standard error.

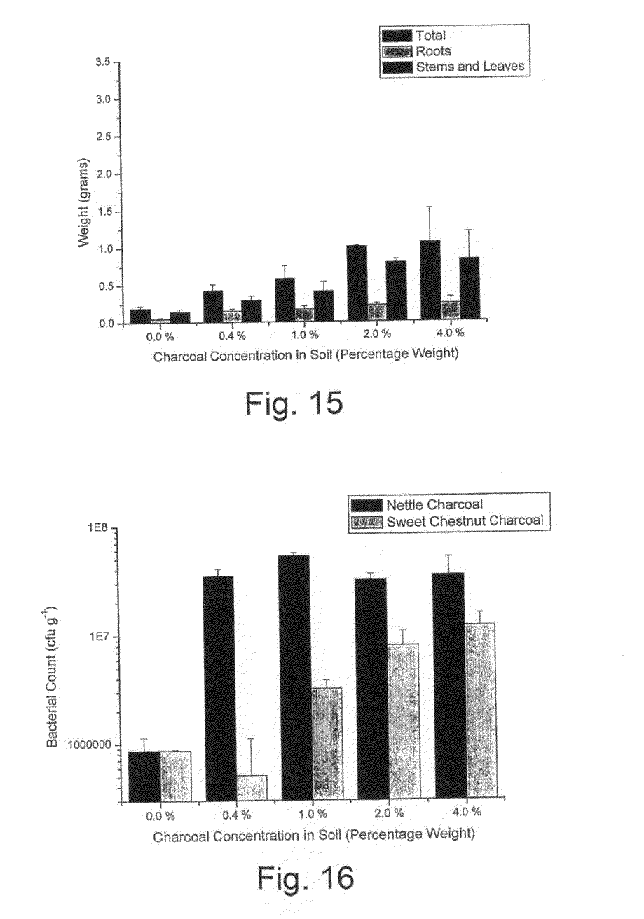

It can be seen that additions of 2 and 4% sweet chestnut charcoal produce significantly increased, root biomass and stem and leaf biomass dry weights after 40 days growth (P<0.05). After 40 days soil amended with sweet chestnut charcoal produced plants that were between 2 and 5.5× heavier than those of the control. FIG. 15 shows sunflower dry biomass after 40 days incubation in soil with different concentrations of sweet chestnut charcoal. N=3. Error bars show standard error.

Conclusions

Amendment of metal contaminated soils with as little as 0.4% (w/w) nettle charcoal restored soil fertility.

Detoxification of soil was possible using wood charcoal, but charcoal produced from stinging nettles was significantly better.

### Example 10

Restoration of Microbial Activity in Metal Contaminated Soil after Amendment with Charcoal

Methodology

Flasks were set up in triplicate with 200 g of each soil combination. 250 cm³ conical flasks were used. To each flask, 2 g wheat straw was added to act as a carbon source. A mixed soil bacterial community was created by mixing a 25 g sample of fresh garden soil with 225 cm³ Ringer's solution and shaken for 30 mins at 150 rpm. The soil suspension was allowed to settle for 20 mins then the supernatant was drawn off. A 5 cm³ sample of soil bacterial suspension was added to each flask. All flasks were sealed with gas exchange bungs to retain moisture but allow gas movement. Flasks were incubated at 20° C. for 36 days. Flasks were left for 24 hours to stabilise, after which they were periodically analysed for CO₂ production/hour using an ADC 225 Mk3 CO₂ analyser. After 18 days 2 g of slow release fertiliser was added to each flask to provide extra nutrients. After 36 days 1 g material from each flask was mixed with 9 cm³ Ringer's solution and shaken to create a bacterial suspension. Bacterial suspensions were diluted and plated onto 1 Tryptone Soya Agar and incubated at 20° C. Counts per gram material were determined.

Results

All nettle charcoal additions increased bacterial counts 100 fold after 40 days growth (P<0.01) compared with the non-amended control. The results are shown in FIG. 16, which shows soil bacterial counts after 40 days of growing sunflowers in soil amended with different concentrations of nettle and sweet chestnut charcoal. N=3. Error bars show standard error. N=3. Error bars show standard error.

Addition of more than 0.4% (w/w) charcoal did not result in greater bacterial numbers. An addition of 2% (w/w) sweet chestnut charcoal was required, in order to produce significantly increased bacterial counts after 40 days growth (P<0.05). Even an amendment of 4% (w/w) with sweet chestnut charcoal only resulted in a 10 fold increase in microbial numbers compared with the non-amended control.

Conclusion

Addition of small quantities (0.4% w/w) of nettle charcoal restored microbial activity in metal contaminated soil.

### Example 11

Differences in Metal Adsorption between Charcoals Derived from Different Tree Species is Related to the Ash Content of the Wood

To investigate whether any difference existed between different species of trees in relation to Cation Exchange Capacity (CEC), charcoals derived from different tree species were screened for their ability to adsorb Cu ions.

Brief Methodology

Eleven different tree species were selected that are commonly grown in the UK for commercial purposes. These were: Sweet chestnut (Castanea sativa), Oak (Quercus robur), Ash, Beech (Fagus sylvatica), Birch (Betula pendula), Eucalyptus (Eucalyptus spp), Crack Willow (Salix fragilis), Poplar (Poplus spp), Alder (Alnus glutinosa), Scots Pine (Pinus silvestrus) and Spruce (Picea abies). Branches or stems with a diameter of around 7 cm were chosen for the experiment. Each branch/stem was sawn into 30 cm lengths and the wood was dried at 25° C. before being charred at 450° C. Each batch of charcoal was divided into 6 sub-samples; three of which were ashes at 600° C. and the other three were ground in a pestle and mortar to determine their ability to adsorb Cu ions.

To determine maximum copper adsorption of each charcoal type, 0.5 g of finely grounded sub-sample of charcoal was suspended in a solution of 250 ml CuSO₄ that contained 250 mg CuSO₄ per 1. Charcoal was kept in suspension using an electric stirrer. Each flask contained excess Cu in relation to the amount of charcoal that could be adsorbed by the suspended charcoal. After 48 hours the charcoal was filtered out, rinsed and digested in concentrated nitric acid. The amount of Cu adsorbed was assessed using Atomic Adsorption (AA).

Results

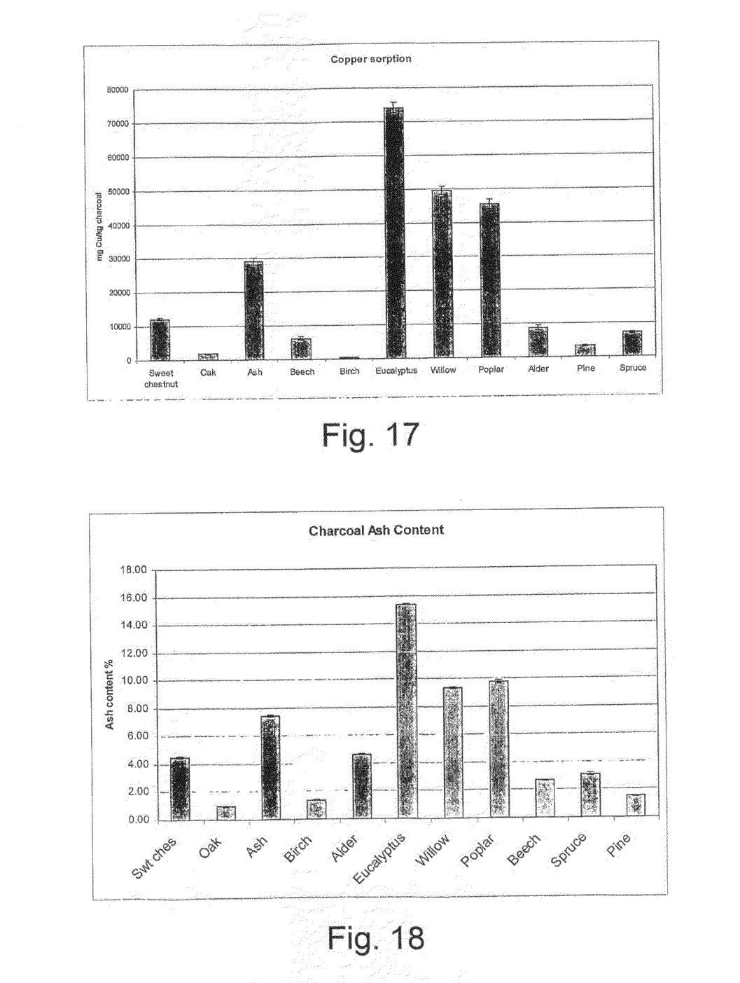

The results are shown in FIGS. 17-19, where:

FIG. 17: Maximum metal adsorption of charcoals derived from 11 different tree species. Branches/stems with a diameter of 7 cm were charred at 450° C. (n=3).

FIG. 18: Ash content of charcoals derived from 11 different tree species. Branches or stems with a diameter of 7 cm were ashed at 600° C. (n=3).

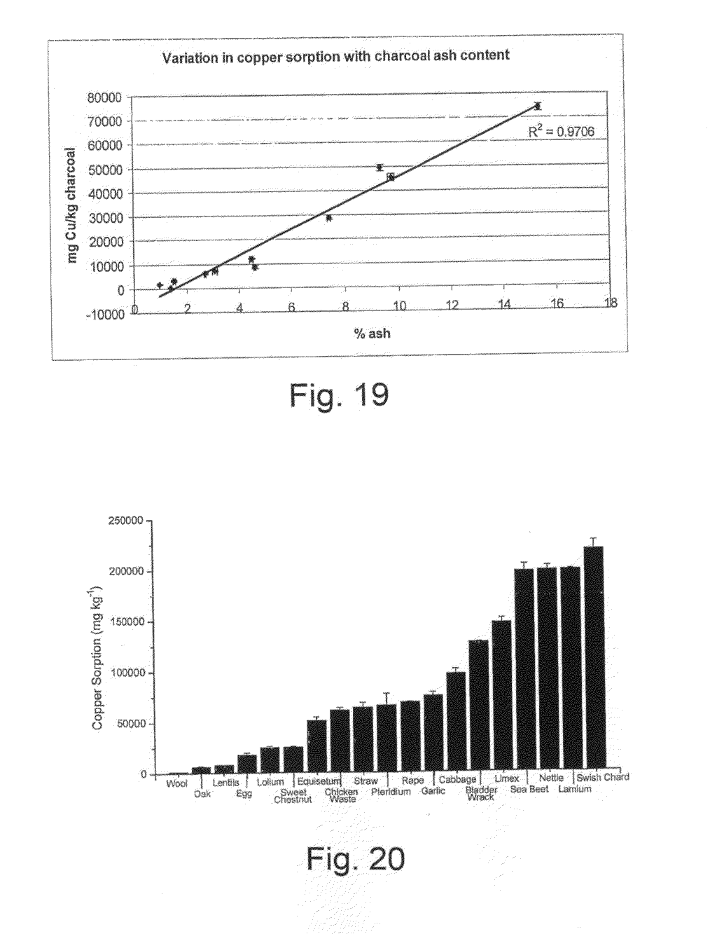

FIG. 19: Correlation between metal adsorption of charcoal and its ash-content (n=3).

Conclusions
Metal adsorption of wood charcoals is strongly correlated to the ash (mineral) content of the charcoal
Relation between Cu adsorption (A) and mineral content (M) on a weight basis is: M=2A
If the exchanged ions are mono-valent and had the same molecular weight of Cu then all ions contained in wood charcoal are exchangeable.
This is not the case as the most common minerals in plants (K and Ca) are 2/3 of the weight of Cu suggesting that not all minerals are exchanged.

See example 18 for further information on this.

### Example 12

Non-Woody Plant Charcoals are also very Effective at Binding Metal Ions, such as Copper.

Brief Methodology

A range of charcoals derived from woody and non-woody plants as well as charcoals derived from chicken litter and lime mixed with sugarbeet impurities (LIMAX) were assessed for their ability to adsorb heavy metals. Three samples of each material were charred at 450° C. To determine the maximum copper adsorption of each charcoal type, 0.5 g of finely grounded charcoal was suspended in a solution of 250 ml CuSO₄ that contained 250 mg CuSO₄ per L. Charcoal was kept in suspension using an electric stirrer. Each flask contained excess Cu in relation to the amount of charcoal that could be adsorbed by the suspended charcoal. After 48 hours the charcoal was filtered out, rinsed and digested in concentrated nitric acid. The amount of Cu adsorbed was assessed using Atomic Adsorption (AA).

In a separate experiment the adsorbing capacity of sugar beet tops was assessed by exposing charcoal produced from sugar beet leaves to increasing concentrations of Cu ions and measure the capacity of the charcoal to remove the Cu from solution. Sugar beet leaves were harvested and dried at 70° C. for 48 hours. Subsequently the material was charred at 450° C. A langmuir isotherm experiment was setup by mixing 0.5 g charcoal samples in 250 ml Cu solution at a range of concentrations from 0 mg/l to 1000 mg/l. After reaching equilibrium samples were filtered and the ability of the charcoal to remove Cu from solution assessed using Atomic Adsorption (AA).

Results

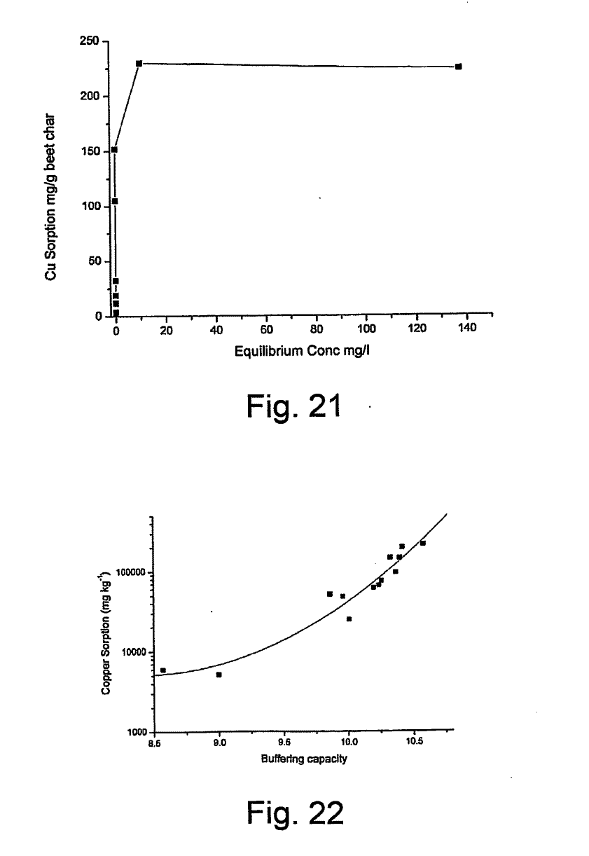

The results are shown in FIGS. 20 and 21, where:

FIG. 20. Copper adsorption onto a range of charcoals derived from woody and non-woody materials (n=3); and

FIG. 21: Langmuir curve describing the ability of charcoal derived from sugar beet leaves to remove Cu ions from solution.

Conclusions
Charcoals derived from non-woody plant materials can be extremely effective at binding heavy metals.
Particularly effective at binding heavy metals are beet (sea-beet, sugar-beet and chard), nettle (deaf nettle and stinging nettle) as well as seaweed (bladder wrack)
Adsorption of these charcoals is 180,000 and 225,000 ppm Cu or between 3 and 3.75 mol Cu per kg charcoal
Below the saturation value of the charcoal all metals are removed from solution.

### Example 13

Ability of Charcoals Derived from Different Source Materials to Raise the pH of Water.

Brief Methodology

The ability of a material to raise the pH of distilled water is a good measure of the CEC (Cation Exchange Capacity) of that material. For the purpose of these experiments, a range of organic materials were selected, known to have a range of metal sorption capacities when charred. Samples of each material were charred at 450° C. Each sample was divided into 6 portions; three for estimating Cu adsorption and three for measuring the ability of the charred material to raise the pH of water.

For measuring metal adsorption, 0.5 g of finely grounded charcoal was suspended in a solution of 250 ml CuSO₄ that contained 250 mg CuSO₄ per L. Charcoal was kept in suspension using an electric stirrer. Each flask contained excess Cu in relation to the amount of charcoal that could be adsorbed by the suspended charcoal. After 48 hours the charcoal was filtered out, rinsed and digested in concentrated nitric acid. The amount of Cu adsorbed was assessed using AA.

To determine the ability of charcoal to raise the pH of de-ionised water, three 0.5 g samples of each charcoal type were suspended in 100 mls RO (Reverse Osmosis) water and the pH of the suspension was measured after equilibrium had been reached. Sorption capacity of each charcoal was thus correlated against buffering capacity, which was used as an indication of its cation exchange capacity (CEC).

Results

The results shown in FIGS. 22 - 24, where:

FIG. 22: Relation between Cu adsorption and ability to raise the pH of water of charcoals derived from different source materials including sweet chestnut, oil seed rape, bladder wrack, sea beet and stinging nettle; and

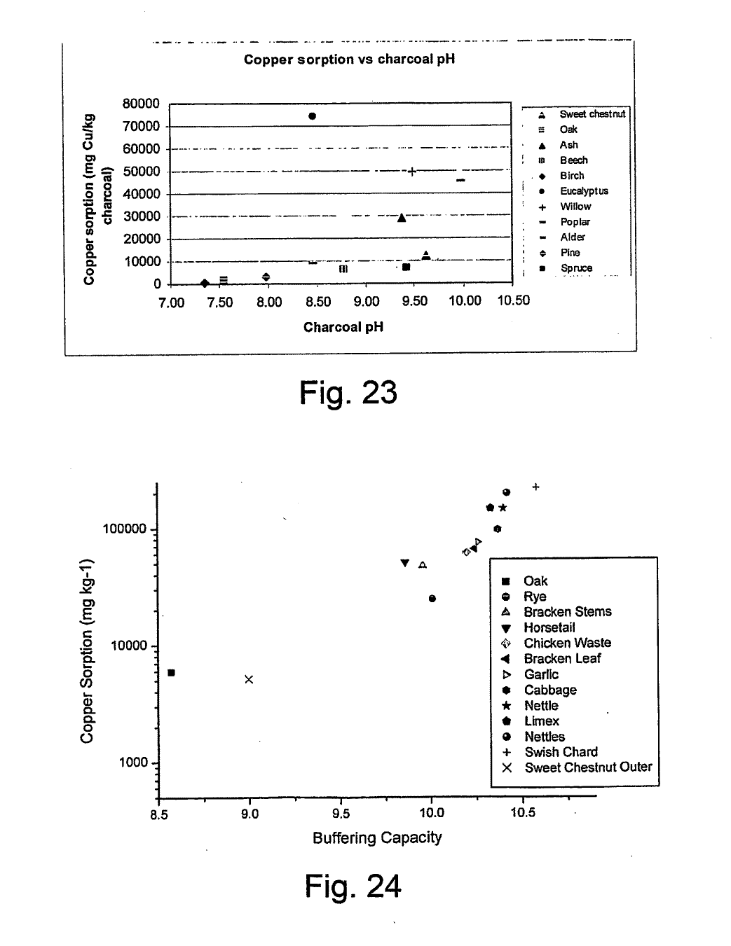

FIG. 23: Relation between Cu adsorption and ability to raise the pH of water of charcoals derived from different tree species.

FIG. 24: Relation between Cu adsorption and ability to raise the pH of water of charcoals derived from different woody and non-woody plant species. The data for FIG. 24 is presented in Table 5 below.

TABLE 5
pH buffering capacity of various plant species.

| | Buffering | Cu Sorption |
|---|---|---|
| Source material | Capacity (pH) | (mg kg⁻¹) |
| Oak | 8.57 | 5980 |
| Sweet Chestnut Outer | 9.00 | 5173 |
| Horsetail | 9.86 | 51067 |
| Bracken Stems | 9.96 | 47670 |
| Rye | 10.01 | 24770 |
| Chicken Waste | 10.20 | 61400 |
| Bracken Leaf | 10.24 | 66000 |
| Garlic | 10.26 | 75000 |
| Cabbage | 10.37 | 96433 |
| Stinging Nettles | 10.42 | 198000 |
| Swiss Chard | 10.58 | 218033 |

Conclusions
There is a good relationship between the ability of charcoal to raise the pH of water and its ability to adsorb metal ions
All charcoals derived from nettle and beet raised the pH of water to between 10 and 11.
None of the charcoals derived from tree species raised the pH above 10.0, whereas the Nettles and Swiss Chard, in particular were able to raise the pH to well above pH 10.0.

### Example 14

Specific Minerals in Charcoal and Metal Adsorption

Brief Methodology

It was hypothesised that young wood is more metabolically active than old wood and that younger wood therefore contains a higher proportion of minerals that are responsible for protein synthesis and photosynthesis. If such minerals are retained after charring, and if they are present in an exchangeable form, this could result in charcoals with a high CEC which have a better ability to adsorb heavy metal ions. To test this hypothesis, sweet chestnut wood of different ages was charred and the mineral content of the resulting charcoals was determined. These data were subsequently correlated with the ability of these charcoals to adsorb Cu and Zn ions from solution.

Going from the outside towards the inside of a tree trunk the wood will become progressively older. To obtain woods of different ages a large tree trunk measuring approx 20 cm in diameter was used. The bark and cambium were removed and the remaining wood was split along the annual lines into sapwood (1-3 years old) outer heartwood (4-6 years) and finally inner heartwood and pith (7-10 years). From each of the four sections 3 portions were separately charred using the methods described.

A branch of a tree will grow both in length and width and each year a new section of wood is added. This means that the top section of a branch represents wood that is less than 1 year old, the section below that is between 1 and 2 years (average 1.5), the one below that between 1 and 3 years (average 2 years), etc. By dividing a branch in ‘year section’ it is possible to obtain wood with a different average age. A large branch measuring approx 7 meters in length was thus divided into 1 m sections. In this way, wood of different ages was obtained ranging from less than 1 year (top of the branch) to sections that were about 2.5 years old on average. Subsequently, from each section including the bark, 3 portions were separately charred using the method described before.

Samples were ground to a fine charcoal powder (<0.5 mm), and a standard batch sorption experiment was set up using 0.5 g charcoal in 250 cm³ metal solution. Solutions contained 250 mg l⁻Cu²⁺ or 250 mg l⁻¹ Zn²⁺ both dissolved as metal sulphates. Samples were shaken for 48 hours. Ashed and acid digested charcoal samples were analysed by Atomic Adsorption (AA) for Cu and Zn. Each sample used for metal adsorption was also analysed by Inductively Coupled Plasma Optical Emission Spectroscopy (ICP-OES) for different minerals to determine if the metal sorption capacity correlated with the elemental composition of the charcoal.

Whereas only one trunk and one branch was analysed, each section was divided into three portions and each portion was charred and analysed separately using analysis of variance.

Results

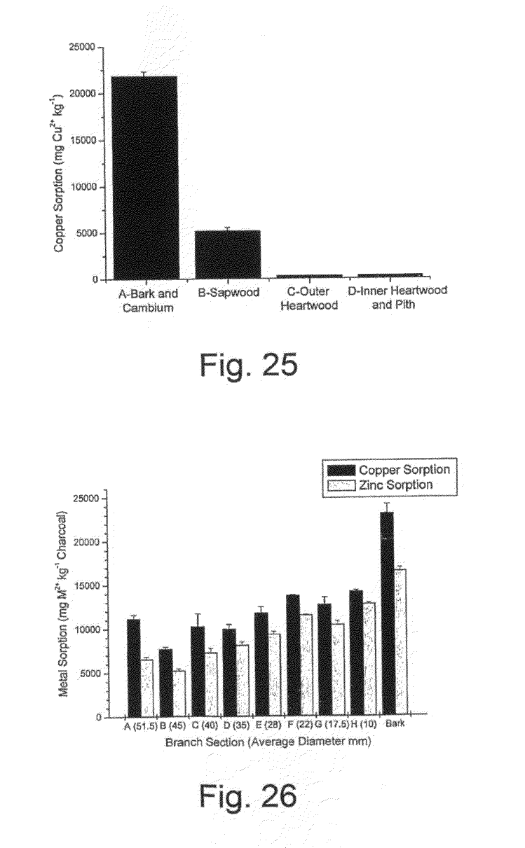

The results are shown in FIGS. 25-30 and Table 6, where:

FIG. 25: Sorption of copper by charcoals produced from sweet chestnut wood of different age. Sections A to D represent sections of a large 20 cm diameter Sweet Chestnut trunk; Section D represents therefore the oldest heartwood and pith while section A is the young bark wood and cambium of <1 year old. All samples were dried, and then charred at 450° C. Charcoal particles were suspended for 48 hours in metal solutions containing Cu²⁺ at 250 mg l⁻¹ N=3;

FIG. 26: Sorption of copper by charcoals produced from sweet chestnut wood of different ages. Sections A (bottom of the branch) to H (top of the branch) represent 1 m sections that become progressively younger. The oldest wood in section A is on average2.5 years old, while section H is wood of <1 year old. Bark was analysed separately. All samples were dried, and then charred at 450° C. Charcoal particles were suspended for 48 hours in metal solutions containing Cu²⁺ at 250 mg l⁻¹ or Zn²⁺ at 250 mg rt. N=3;

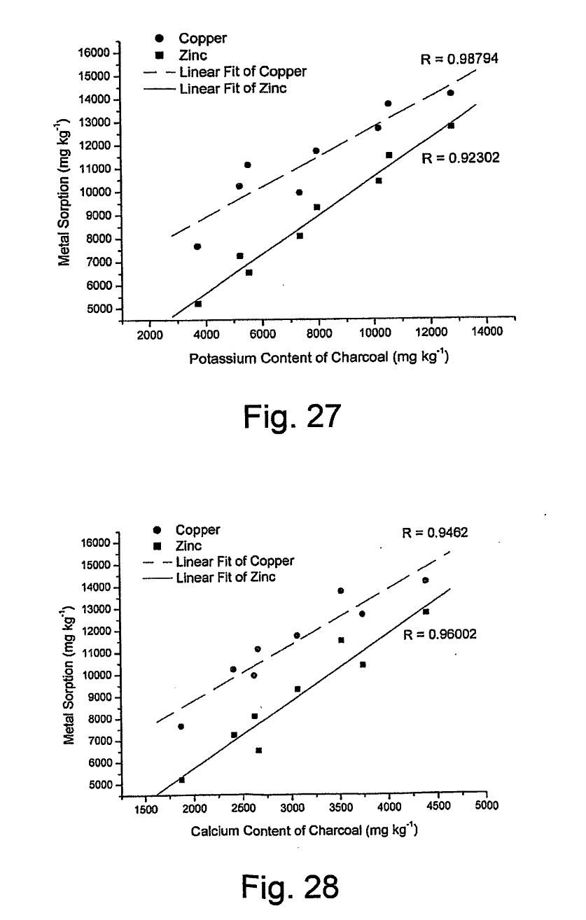

FIG. 27: Correlation between of maximum Copper and Zinc sorption onto charcoal and the concentration of Potassium in charcoal before exposure to Cu ions;

FIG. 28: Correlation between of maximum Copper and Zinc sorption onto charcoal and the concentration of Calcium in charcoal before exposure to Cu ions;

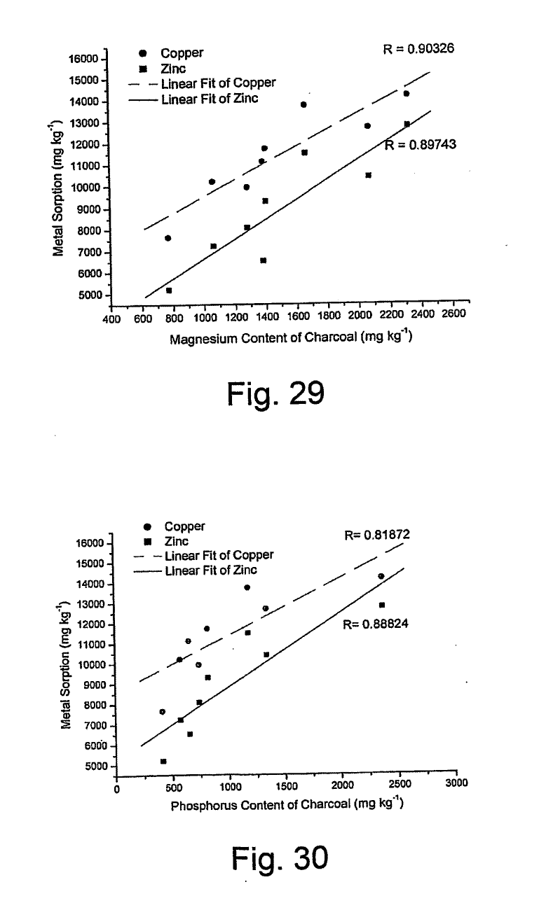

FIG. 29: Correlation between of maximum Copper and Zinc sorption onto charcoal and the concentration of Magnesium in charcoal before exposure to Cu ions; and

FIG. 30: Correlation between of maximum Copper and Zinc sorption onto charcoal and the concentration of Phosphorus in charcoal before exposure to Cu ions.

TABLE 6
Mean mineral concentration (mg kg⁻¹ and mM) in charcoals produced from
sweet chestnut wood of different ages. .

| | Mean Concentration in Charcoal | | Correlation (R) | |
|---|---|---|---|---|
| Element | (mg kg⁻¹) | (mM) | Zn | Cu |
| K | 7908.75 | 202.27 | 0.988 | 0.923 |
| Ca | 3033.75 | 75.65 | 0.960 | 0.946 |
| Mg | 1492.50 | 62.42 | 0.897 | 0.903 |
| P | 1010.00 | 32.58 | 0.888 | 0.819 |
| Mn | 384.42 | 7.00 | 0.883 | 0.838 |
| Na | 97.13 | 4.22 | 0.466 | 0.524 |
| Al | 67.70 | 2.51 | 0.948 | 0.861 |
| Fe | 57.59 | 1.03 | 0.895 | 0.848 |
| B | 21.75 | 2.01 | 0.852 | 0.847 |
| Ni | 1.73 | 0.03 | 0.767 | 0.756 |
| Cd | 0.20 | 0.00 | 0.543 | 0.693 |
| Cr | 0.17 | 0.00 | 0.442 | 0.585 |
| Co | 0.14 | 0.00 | −0.220 | −0.040 |

Correlation is against Zn²⁺ and Cu²⁺ sorption by the same charcoals after they were suspended for 48 hours in metal solutions containing Cu²⁺ at 250 mg l⁻¹ or Zn²⁺ at 250 mg l⁻¹ (N = 3).
Mean Cu²⁺ sorption was 11407.75 mg kg⁻¹ (179.60 M).
Mean Zn²⁺ sorption was 8871.00 mg kg⁻¹ (135.60 M).

Conclusions
Charcoals produced from ‘metabolically active’ wood (bark and sapwood) are more adsorbent to heavy metals than ones produced from non-active wood
The most abundant mineral in (wood) charcoal is Potassium (63% of total mineral content) followed by Calcium (23% of total mineral content), Magnesium (11% of total mineral content), Manganese (3% of total mineral content). Al other minerals (Na, Al, B, Ni) represent <1% of the total mineral content
There are good correlations between the mineral content of charcoal and ability to adsorb metals
Strongest correlation with metal adsorption are with K, Mg and Ca (R²>0.9) as well as P (R²=0.8)
For every P there are 5-10 metal ions adsorbed suggesting that adsorption onto phosphate groups represents a minor component in the metal adsorption of charcoal
Cations such as K, Mg and Ca could be exchanged for metal ions—phosphate could be a functionally binding group on the charcoal surface

### Example 15

Exchange of Minerals and Metal Adsorption

Brief Methodology

In order to prove that metal adsorption could be explained by exchange of cationic minerals present in charcoal 5 different source materials were chosen. Each material, when charred has a different capacity to adsorb heavy metals: In order of capacity to adsorb metals these materials were derived from a sweet chest nut branch, oilseed rape plants, bladder wrack, stinging nettle and sea-beet leaves. Charcoal derived from sea-beet leaves had the greatest ability to adsorb metals and charcoal derived from sweet chestnut adsorbed least metals. For each material samples were harvested from three separate sites. After harvesting materials were dried at 70° C. for 7 days. Each samples was ground and homogenised to create an even mix with <2 mm particle size.

Subsequently a 50.0 g samples of each material was charred at 450° C. Weight of charcoal produced was measured and thus charcoal yield per gram dry weight plant matter could be calculated.

Samples of 0.5 g charcoal were then suspended in a 250 ml solution of CuSO₄ containing 250 ppm Cu. Duplicate samples for each charcoal sample were suspended for 48 h in this solution, before samples were filtered, dried, digested, and analysed by Inductively Coupled Plasma Optical Emission Spectroscopy (ICP-OES) for a range of elements. The dried plant matter and untreated charcoals were also analysed allowing loss of ions during charring as well as exchange of ions to be calculated. Correlation between ion-exchange and metal adsorption onto the different charcoals was calculated subsequently.

Results

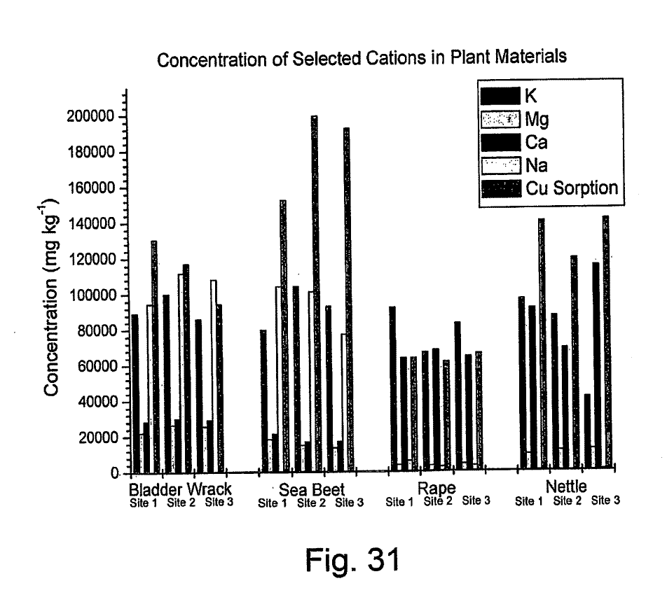

The results are shown in FIGS. 31-35, where:

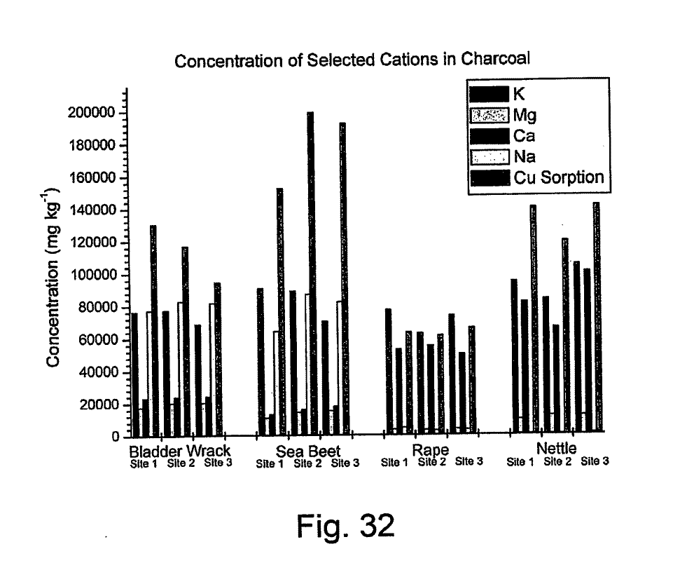

FIGS. 31 and 32: Concentration of key minerals (K, Ca, Mg and Na) in plant material before and after charring in Bladder wrack, Sea beet, oil seed rape and stinging nettle. Concentrations in dried plant material are accounted for loss of weight as a result of charring;

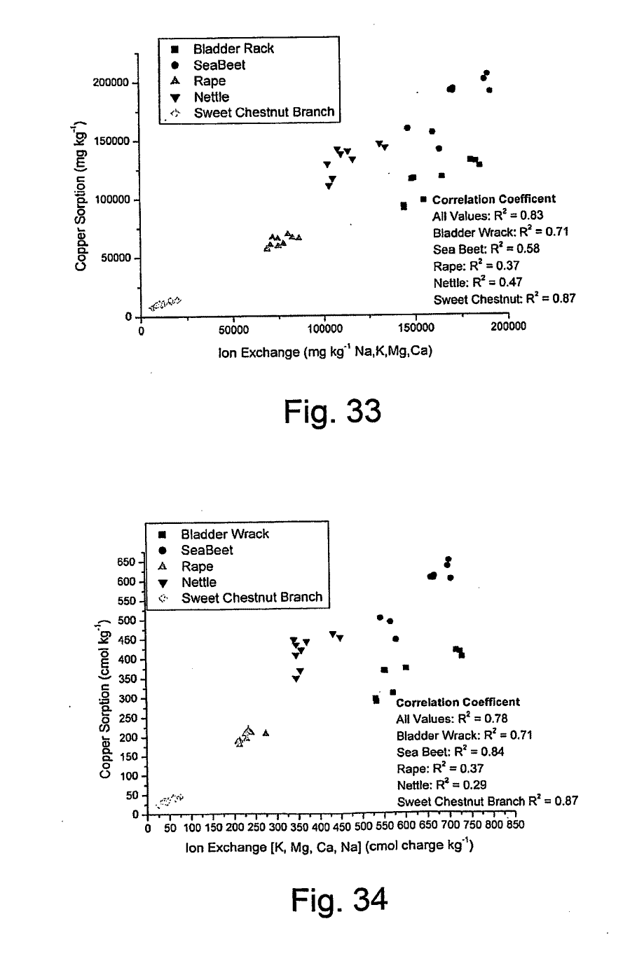

FIG. 33: Correlation between weight of exchanged ions and weight of adsorbed copper ions using charcoals derived from different source materials, including bladder-wrack. Each data point represents a group of plants taken from a particular site;

FIG. 34: Correlation between charge of exchanged ions and charge of adsorbed copper ions using charcoals derived from different source materials, including bladder wrack. Each data point represents a group of plants taken from a particular site; and

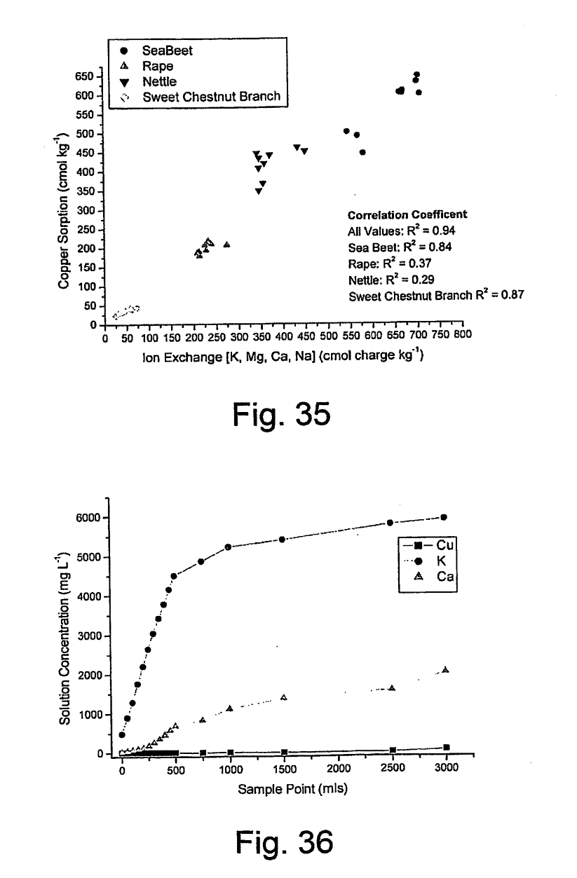

FIG. 35: Correlation between charge of exchanged ions and charge of adsorbed copper ions using charcoals derived from different source materials, excluding bladder wrack. Each data point represents a group of plants taken from a particular site.

Conclusions
Exchange of minerals such as K, Ca, Mg and Na by charcoal explains why certain charcoals are extremely good at adsorbing heavy metals.
Adsorption (A) on a charge (C) basis is A=C
Charring makes the minerals in a specific source material ‘exchangeable’
Soluble salts in the cytoplasm of seaweeds don't contribute to metal adsorption when the material is charred

### Example 16

Sequence of Ion Exchange During Copper Adsorption onto Charcoal

Brief Methodology

One possible use of highly metal adsorbent charcoals is as a filter material in water filters or permeable reactive barrier systems. An experiment was set up to monitor metal removal from a solution containing 500 ppm Cu²⁺ dissolved as CuSO₄ in RO (Reverse Osmosis) water in the first instance. A 5 cm diameter glass column was packed with a 20 g of a 50:50 mixture of charcoal derived from stinging nettle and bladder-wrack. The metal contaminated solution was filtered through this material at a rate of 10 ml per minute. For the first hour, every 5 minutes 10 ml of the filtered solution was collected. For the next hour samples were taken at a half hourly rate. At this point the concentration of Cu in solution was doubled to 1000 ppm and then the sampling regime was reduced to hourly collections. Sampling was continued till Cu started to break through (visible as a blue haze in the solution). In this way 16 samples were collected. Each sample was analysed for Cu (which was to be removed) and exchanged cations (K, Ca, Mg, etc) using Inductively Coupled Plasma Optical Emission Spectroscopy (ICP-OES). Doing this, it was possible to obtain the sequence of ions that were exchanged from the charcoal.

Results

The results are shown in FIG. 36, where:

FIG. 36: Cumulative concentrations of Cu, K and Ca in filtrate from a Cu solution containing 500 ppm Cu²⁺ that was passed through a 5 cm diam. glass column packed with 10 g of a 50:50 mix of charcoal derived from stinging nettle and bladder wrack. (n=1).

Conclusions
The mixture effectively removed Cu from solution
During Cu adsorption, Potassium ions were exchanged first, followed by Ca ions
All other ions (Except Mg) were below the level of detection.

### Example 17

Dependence of Adsorbing Properties of Nettle Charcoal on Growth Conditions of the Plants

Brief Methodology

Stinging nettles (Urtica dioica) were collected from different locations in the South East of England in July 2006. Sites were chosen on the basis of nettle phenotypes that were growing; large (up to 1.5 m high), dark green plants were indicative of high soil fertility, while small (around 0.5 m high), light green plants were indicative of poor soil fertility. The most nutrient rich locations were manure heaps while the most nutrient poor situations that supported nettle growth were on a chalk hill side. Besides the effect of phenotypic variation on metal adsorption, stems and leaves were analysed separately for their metal adsorbing capacity.

Results

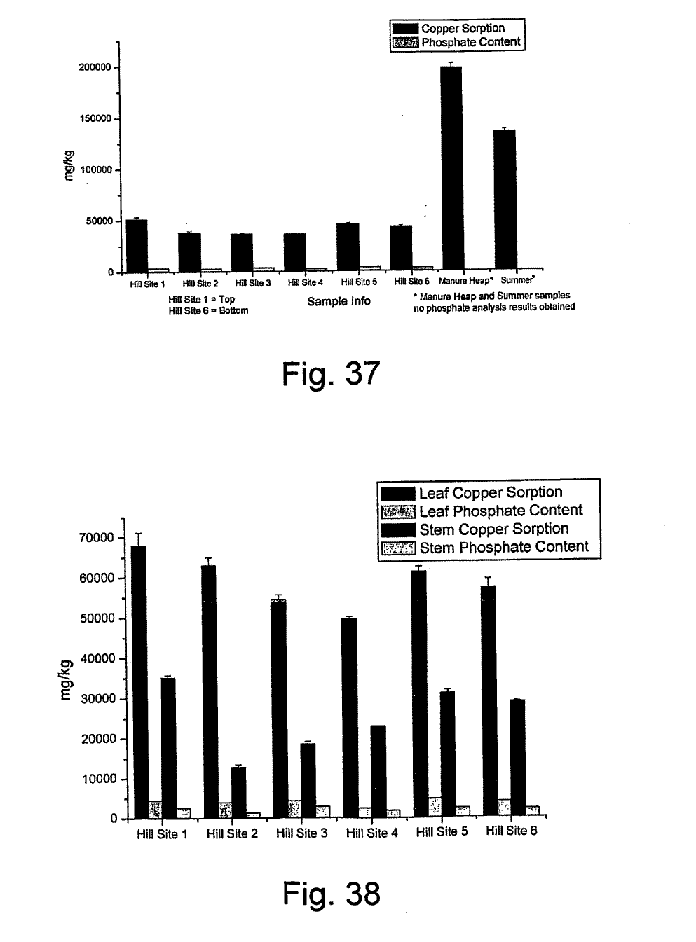

The results are shown in FIGS. 37 and 38, where:

FIG. 37: Adsorption of Cu onto nettle charcoal produced from the leaves and stems of stinging nettles (Urtica dioica) that grew at different locations (Hill side are nettles taken from a chalk hill). N=3; and

FIG. 38: Adsorption of Cu onto nettle charcoal produced from either stinging nettle leaves or stems. All plants were taken from nettle patches that grew on a chalk hill, low in nutrients. N=3.

Conclusions
Plants growing in highly fertile soil can produce charcoal that are four times more adsorbent to metal ions than charcoal produced from plants that grew under nutrient deficient conditions.
Charcoal produced from plant leaves is between 2 to 5 times more adsorbent to metal ions than stems.

### Example 18

Relationship between Ash Content of Non-Woody Plants and Metal Adsorption

Brief Methodology

For 11 different tree species it was established that once the wood was charred, the ash content of the charcoal was strongly correlated to the ability of these charcoals to adsorb heavy metals. The relationship between the ash content of the char and the ability of the char to adsorb Cu was found to be: Ash content=2×Adsorbtion (see example 11).

In this experiment, 11 different source materials were charred at 450° C. These materials included 2 tree species (oak and sweet chestnut), one grass (Rye grass), a fern (Bracken), a macro-algae (bladder wrack), one bulb (garlic), oil seed rape, stinging nettle stems and leaves and sea beet leaves. Of these, ryegrass are known to contain a large amount of Si, while bladder wrack has a high (free) sodium concentration in its vacuoles to allow these plants to maintain cell turgor in the salty environment where they grow. To determine the ash content of the different charcoals, 1 g charcoal derived from each of the different plant species was placed in a pre-weighted crucible and heated to 550° C. for 12 hours. Ash content was expressed as a percentage of the original charcoal weight.

Results

TABLE 7

| | Cu Sorption | |
|---|---|---|
| Source material | (mg kg−1) | Ash (%) |
| Oak | 5980 | 1.50 |
| Sweet Chestnut Outer | 5173 | 1.91 |
| Rye | 24770 | 20.90 |
| Bracken Stems | 47670 | 11.13 |
| Rape | 63580 | 32.1 |
| Bracken Leaf | 66000 | 20.19 |
| Garlic | 75000 | 9.38 |
| Cabbage | 96433 | 16.89 |
| Bladder Wrack | 113872 | 54.7 |
| Nettle | 133460 | 43.6 |
| Seabeet | 181304 | 46.6 |
| | RSQ | 0.66 |

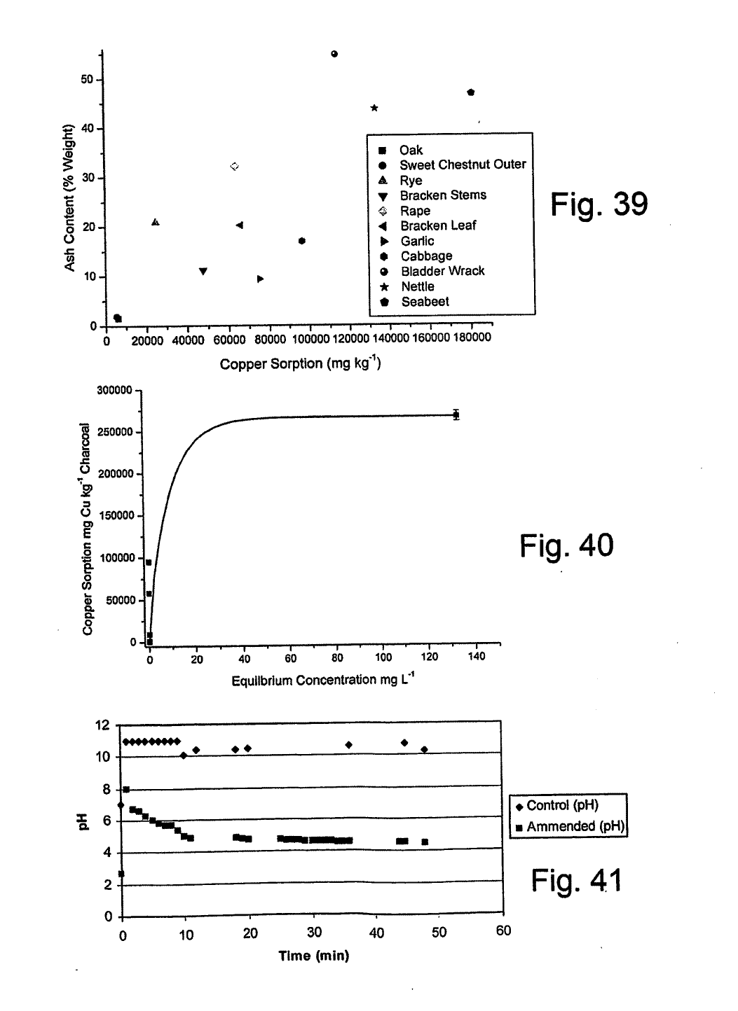

Table 7 above and FIG. 39 show the relation between ash content of charcoals produced from a variety of plants, including woody plants, grass, a fern, a sea weed and a number of dicotyledons (cabbage, beet, garlic, stinging nettle and oil seed rape).

Conclusions
There is a positive correlation (R²=0.66) between ash content of charcoals derived from a wide variety of plants and the ability of these charcoals to adsorb metals.
Ratio between ash content and Cu adsorption is around 3 (M=3A).
An ash content of char greater than 15% indicates a charcoal with metal adsorbent properties.
Free sodium present in plant vacuoles does not contribute to ion exchange.
Si is not important for ion exchange

### Example 19

### Calcium Modified Charcoal

Introduction

We found that potassium is the main exchangeable element of charcoals made from nettle, beet etc. When brought into the environment, potassium is also exchanged with hydrogen ions. In some cases this is an advantage when a high pH is required (for example to allow precipitation of metal ions as metal hydroxides. However, this ability of Potassium to be exchanged with hydrogen is disadvantageous if the pH of the medium needs to be maintained around neutral. Furthermore, hydrogen ions, once adsorbed onto the charcoal are less readily exchanged against heavy metal ions than potassium, making the charcoal less comparable of removing metals from the environment via adsorption.

To overcome this problem we have been able to create a charcoal where potassium is replaced by Ca ions. Other ions such as Mg and Mn could be equally be used in place of Ca ions to achieve the same charcoal properties.

Brief Methodology

13.65 g CaCl2.6H20 (which is 2.5 g Ca ions) was dissolved in 500 cm3 RO water. To this solution 10 g nettle charcoal (<0.5 mm mesh size) was added. The mixture was sealed and stirred using a magnetic stirrer for 48 hours. After this time the charcoal was filtered out using a whatman No. 1 filter paper placed on a large Buchner filter. The charcoal was then dried at 40° C. over night. Metal adsorption and effect on pH were assessed using standard methods as described previously.

Results

The modified charcoal not only has the ability to adsorb 20% more heavy metal ions (250,000 ppm Cu instead of 200,000 ppm), it also does not change the pH of normal tap water by much more than one unit (data not presented). The results are shown in the Langmuir curve presented as FIG. 40 (an adsorption isotherm of Ca-modified nettle charcoal).

### Example 20

### Acidified Charcoals

In most cases raising the pH of the environment is advantageous to reduce metal bio-availability. However other metal ions, notably anionic metals such as As, are mobilized at high pH. Also, ammonium ions are converted into toxic ammonia at high pH. A cheap ion-exchange material that releases hydrogen ions to lower the pH of the medium could be advantageous in media such as animal beddings, where a low pH would prevent the conversion of ammonium to ammonia. The advantage of using acidified charcoals is that these materials are long-lasting and are less reactive under moist conditions than acidic salts such as alum and hydrogen-bisulphate. Other ion-exchange materials such as zeolites are also modified with hydrogen ions to obtain favourable properties, but the process is expensive involving saturation with ammonium ions followed by a heating step to remove ammonium thus leaving exchangeable hydrogen ions. This cumbersome process is necessary for zeolites which dissolve when brought directly into contact with acids—charcoals are stable under acidic conditions and can be used directly to create acidified charcoals.

Besides obtaining a product that has its uses for lowering the pH of the environment, the process can yield substantial quantities of chemical fertilizer. Using Nitric or phosophoric acid, the solution will be converted into a mixture of potassium nitrate, potassium phosphate and a number of other salts containing phosphate and nitrate. These fertilizer salts can be recovered from the solution by evaporation of the excess water.

Experiment A: Ability of Acidified Charcoal to Reduce pH of Spent Chicken Litter and Prevent the Formation of Ammonia

Fresh chicken litter was collected from under a chicken roost. This material consisted of wood shavings and chicken faeces.

To obtain acidified charcoal, finely ground nettle charcoal was treated with 1 molar nitric acid overnight till ca 90% of acid was removed from solution by the charcoal (pH 1). After draining the charcoal the charcoal was dried a 90° C. till dry.

Treatment: 25 g charcoal was amended to 500 g chicken litter and the mixture was moistened with a further 50 ml water to obtain optimal conditions for ammonia production.

Control: no amendment to 500 g litter but moistened with 50 ml water

System: 5 litter closed Dispo-jars. The treated and non-treated litter was slightly compressed and formed a 10 cm layer at the bottom.

Incubation temperature: 30° C.

Results:

Ammonia—Qualitative Assessment

After 3 days the non-treated litter started to smell of ammonia

After 5 days the ammonia smell was quite strong in the non-treated litter

After 11 days ammonia smell was almost gone in the non-treated litter

After 12 days opened vessels to aerate—within hours the non-treated litter started to smell strongly of ammonia (no ammonia smell in the treated litter)

After 14 days (after venting) no smell in either treatment; the litter was fairly dry, so sprayed approx 50 ml water on surface; replaced cap

After 16 days no ammonia smell in either treatment—experiment looks finished pH measurements (using 10 g litter (wet weight) per 40 ml RO water)

TABLE 8
pH in chicken litter treated with 5% (w/w) acidified
charcoal compared with a non-amended control.

| day | non treated | treated |
|---|---|---|
| 3 | 7.9 | 7.5 |
| 5 | 8.5 | 7.0 |
| 11 | 8.3 | 6.65 |
| 14 | 7.6 | 6.06 |
| 16 | 7.0 | 6.12 |

Follow Up Experiment

Clearly most of the convertible nitrogen had disappeared after 14 days. To challenge the system further, 3.5 g urea was added on day 16 of the experiment.

Results

Qualitative Assessments

3 h after addition: Strong ammonia smell in control; no smell in treated system Day 1 (24 h after amendment with urea) Overwhelming smell of ammonia in control; faint ammonia smell in treatment

Day 4 Both control and treatment smelled faintly of ammonia

pH Measurements in Continued Experiment

TABLE 9
pH in chicken litter treated with 5% (w/w) acidified
charcoal compared with a non-amended control after an
amendment with 3.5 g urea per 500 g chicken litter

| Day after urea amendment | non treated | treated |
|---|---|---|
| Day 1 | 8.9 | 7.8 |
| Day 4 | 7.8 | 7.7 |

Experiment B: Ability of Acidified Charcoal to Reduce pH of an Ammonium Solution

In a follow-up experiment the ability of acidified charcoal to lower the pH of an ammonium/ammonia solution was assessed by adding 1 g charcoal to 100 ml of ammonia solution. The effect of acidified nettle charcoal on the pH of an ammonium solution is shown in FIG. 41.

In FIG. 41 it can be clearly see there is a large difference between the control and the charcoal amended treatment. Before addition of ammonia the charcoal amended treatment had a pH of 3 and the non-amended treatment (RO water) had a pH of 7. The addition of the ammonia caused an increase in the pH to a value of around 11 of the non-amended treatment while the pH of the charcoal amended treatment did rise to 7 immediately after ammonium amendment. Subsequently, the pH in the charcoal amended systems dropped within 10 minutes to a pH of 4.3. Two days later the pH in the amended systems stabilised at a pH of 3.82, whereas the control had a pH of 10.56.

### REFERENCES

Antal. M. J and Gronli, M. (2003) The art, science and technology of charcoal production. Industrial and Engineering Chemistry Research, 42, 1619-1640.
Baird, C. and Cann, M. (2005) Environmental Chemistry, 3rd edn, Freeman, New York.
Lima, I. M. and Marshall, W. E. 2005. Adsorption of Select Environmentally Important

Metals by Poultry Manure-Based Granular Activated Carbons. Journal of Chemical Technology and Biotechnology. 80, 1054-1061.
Knox, A. S, Kaplan, D. I. and Paller, M. H. (2006) Phosphate sources and their suitability for remediation of contaminated soils. Science of the Total Environment, 357, 271-279.
Machida, M., Yamzaki, R, Aikawa, M. And Tatsumoto, H. (2005) Role of minerals in carbonaceous adsorbents for removal of Pb(II) ions from aqueous solution. Separation Purification Technology, 46, 88-94.
Niyogi, S., Abraham, T. E. and Ramakrishna, S. V. (1998) Removal of chromium (VI) ions from industrial effluents by immobilised biomass of Rhizopus arrhizus. Journal of Scientific and Industrial research, 57, 809-816.
Norris, P. R. and Kelly, D. P. (1977) Accumulation of cadmium and copper by Saccharomyces cerevisiae. Journal of General Microbiology, 99, 317-324.
Tobin, J. M., Cooper, D. G. and Neufield, R. J. (1990) Investigations of the mechanism of metal adsorption by Rhizopus arrhizus biomass. Enzyme and Microbial Technology, 12, 591-595.

1. An ion exchange agent for adsorbing cations, said agent comprising charred material, wherein said charred material is not activated and is produced from living plant material that is not wood, having an ash content of at least 15% (by weight).

2. The agent of claim 1, wherein said material is foliage and said cations are heavy metal cations.

3. The agent of claim 1, wherein said charred material is produced from plant tissues that are less than one year old at said time of harvest.

4. The agent of claim 1, wherein said material is not wood or secondary xylem material.

5. The agent of claim 1, wherein said living material is metabolically active at the time of harvesting.

6. The agent of claim 1, wherein said material is nettle, beet, an algae or seaweed.

7. The agent of claim 6, wherein said plant is a macro algae or seaweed, selected from the group consisting of: bladder wrack (Fucus spp), oarweeds/kelp (Laminaria spp), thongweed (Hinanthalia spp) and sea lettuce (Ulva spp) .

8. The agent of claim 1, wherein said plant material is from an herbaceous plant or a crop.

9. The agent of claim 8, wherein said plant material is selected from the group consisting of: nettle (Urticae spp), dead nettle (Lamium spp), Chenopodiaceae.

10. The agent of claim 1, wherein said plant material is selected from the group consisting of cabbage, garlic, bracken, horsetail, rye grass and oil seed rape.

11. (canceled)

12. The agent claim 1, wherein said ash content of said charcoal is at least 20% (by weight).

13. The agent of claim 1, wherein said ash content of said charcoal is at least 25% (by weight).

14. The agent of claim 1, wherein said ash content of said charcoal is at least 30% (by weight).

15. The agent of claim 1, wherein said ash content of said charcoal is at least 35% (by weight)

16. The agent of claim 1, wherein said ash content of said charcoal is at least 40% (by weight).

17. The agent of claim 1, wherein said ash content of said charcoal is at least 45% (by weight).

18. The agent of claim 1, wherein said ash content of said charcoal is at least 50% (by weight).

19. The agent of claim 1, wherein K, Ca, Mg, Mn and/or P make up at least 10% of said charcoal weight.

20. The agent of claim 1, wherein K, Ca, Mg, Mn and/or P make up at least 15% of said charcoal weight.

21. The agent of claim 1, wherein K, Ca, Mg, Mn and/or P make up at least 20% of said charcoal weight.

22. The agent of claim 1, wherein K, Ca, Mg, Mn and/or P make up at least 25% of said charcoal weight.

23. The agent of claim 1, wherein K, Ca, Mg, Mn and/or P make up at least 30% of said charcoal weight.

24. The agent of claim 1, wherein K, Ca, Mg, Mn and/or P make up at least 35% of said charcoal weight.

25. The agent of claim 1, wherein K. Ca, Mg, Mn and/or P make up at least 40% of said charcoal weight.

26. The agent of claim 1, wherein 0.5 g of charred material is capable of raising said pH of 100 ml deionised water to a pH of at least 10.

27. The agent of claim 1, wherein said charred material adsorbs cations from a selected environment.

28. The agent of claim 27, wherein said cations are selected from the group consisting of: ammonium, copper, zinc, lead, mercury, nickel, cadmium, mercury and aluminium.

29. The agent of claim 27, wherein said environment or area for treatment is soil or an aqueous waste, selected from waste water or sewage.

30. Animal bedding or clothing comprising an agent of claim 1.

31. A method for removing a cationic dye from a solution, said method comprising contacting an agent any of claim 1 with said solution.

32. A filter comprising an agent of claim 1.

33. A composting enhancer or accelerator comprising an agent of claim 1.

34. A cosmetic product comprising an agent of claim 1.

35. A plant growth medium comprising an agent of claim 1.

36. A method for the removal or binding of cationic species in an environment, said method comprising contacting said cationic species with an agent of claim 1.

37. The method of claim 36, wherein said cationic species is one or more metal species.

38. The method of claim 36, wherein said environment is soil, solid waste, a slurry or an aqueous waste.

39. The method of claim 36, wherein treatment of said environment is effected by trapping said agent in a vehicle and passing a liquid over or through said vehicle, thereby to contact said trapped charcoal and permit removal of some or all of said contaminating cations.

40. A method for treating or remediating an environment, comprising contacting said area with an agent of claim 1, and optionally subsequently removing said agent.

41. A method to raise the apparent pH of acidic soil toward pH 7, said method comprising contacting said soil with an agent of claim 1 in an amount and for a period sufficient to elevate said pH of said soil.

42. A process for providing an ion-exhange agent of claim 1, where living plant material containing non-exchangable ions is charred, thereby providing said an ion-exchange agent.

43. The ion exchange agent of claim 1, modified after charring, wherein naturally occurring potassium ions are replaced by other suitablecations selected from Calcium, Manganese Magnesium, or Hydrogen ions.
